(19) United States
(12) Patent Application Publication
(10) Pub. No.: US 2023/0207123 A1
(43) Pub. Date: Jun. 29, 2023

(54) MACHINE LEARNING APPROACH FOR DETECTING DATA DISCREPANCIES DURING CLINICAL DATA INTEGRATION

(71) Applicant: GE Precision Healthcare LLC, Milwaukee, WI (US)

(72) Inventors: Sagi Schein, Kiryat Tivon (IL); Gary Van Nicolas, Valparaiso, IN (US); Ruth Bergman, Ceasarea (IL); William S. Felski, Merritt Island, FL (US); Ole Jakob Utkilen, Tel Aviv (IL)

(21) Appl. No.: 17/645,901

(22) Filed: Dec. 23, 2021

(51) Int. Cl. G16H 50/20; G16H 70/20

(52) U.S. Cl. CPC G16H 50/20; G16H 70/20

(57) ABSTRACT

Techniques are described that employ a machine learning approach for detecting data discrepancies during clinical data integration. In an embodiment, a computer implemented method comprises receiving historical clinical data messages converted from a native format to a target format via a mapping function that maps different sets of historical data elements included in the historical clinical data messages into defined data description paths, and training anomaly detection models for each of the defined data description paths to characterize normal characteristics of the different sets of historical data elements for each of the defined data description path. The method further comprises receiving new clinical data messages converted from the native format or to the target format via the mapping function, and detecting abnormal characteristics of different sets of new data elements for corresponding data description paths of the defined data description paths using the anomaly detection models.

### TECHNICAL FIELD

This application relates to a machine learning approach for detecting data discrepancies during clinical data integration.

### BACKGROUND

Many clinical applications used in active hospital environment consume clinical data exported from various disparate electronic clinical data information sources. For example, in a hospital environment, multiple electronic information systems typically stream such data through a single gateway which standardize it into a single data feed for processing by clinical applications. In these environments, many of the clinical information systems were never designed to easily export data to external consumers. For example, some information systems may export data in different versions of the Health Level Seven (HL7™) format, while others may use proprietary file formats or external databases to export data. To handle these data formatting discrepancies uniformly, the data feeds are typically mapped through a data mapping solution into a single canonical data format. For example, data received in various native formats may be mapped to the Fast Healthcare Interoperability Resources (FHIR™) through a data mapping solution via a set of mapping rules in the form of translation tables and associated scripts. However, constructing these mapping rules is a manual, time consuming and error prone process that requires technical expertise of an integration engineer. In addition, the mapping rules are tailored for each integration project to account for the specific set of clinical information systems involved and the formatting requirements of the specific consuming application or applications. Furthermore, the mapping rules must be routinely validated by clinical experts, debugged and updated to account for changes in the system, such as addition and removal of information sources and software changes to the information sources. Accordingly, techniques for improving the efficiency of the clinical data integration process are in high demand.

### SUMMARY

The following presents a summary to provide a basic understanding of one or more embodiments of the invention. This summary is not intended to identify key or critical elements or delineate any scope of the different embodiments or any scope of the claims. Its sole purpose is to present concepts in a simplified form as a prelude to the more detailed description that is presented later. In one or more embodiments, systems, computer-implemented methods, apparatus and/or computer program products are described that provide a machine learning approach for detecting data discrepancies during clinical data integration.

According to an embodiment, a system is provided that comprises a memory that stores computer executable components, and a processor that executes the computer executable components stored in the memory. The computer executable components comprise a machine learning component that receives historical clinical data messages converted from one or more first native formats to a target format via a mapping function that maps different sets of historical data elements included in the historical clinical data messages into defined data description paths. The machine learning component trains anomaly detection models for each of the defined data description paths using machine learning to characterize normal characteristics of the different sets of data elements for each of the defined data description paths. The computer executable components further comprise an anomaly detection component that receives new clinical data messages converted from the one or more first native formats or one or more second native formats to the target format via the mapping function and detects abnormal characteristics of different sets of new data elements mapped from the new clinical data messages for corresponding data description paths of the defined data description paths using the anomaly detection models.

In various embodiments, the anomaly detection component applies respective anomaly detection models of the anomaly detection models for the corresponding data description paths to the different sets of new data elements respectively mapped to each of the defined data description paths and generates anomaly scores for each of the corresponding data description paths that represent an amount or severity of the abnormal characteristics associated with each of the corresponding data description paths. The computer executable components further comprise an alert component that generates an integration error alert for any of the corresponding data description paths whose anomaly score exceeds a threshold anomaly score. The computer executable components further comprise a reporting component that generates integration report data identifying the anomaly scores for each of the corresponding data description paths and identifying any of the defined data description paths associated with an integration error alert. In some implementations, the computer executable components further comprise a rendering component that presents the integration report data via a graphical user interface. The graphical user interface can further provide interactive mechanisms that facilitate inspecting the integration report data, including data description paths and representative data samples associated with integration error alert and providing feedback regarding the accuracy of the potential integration errors. The machine learning component can further be configured to regularly retrain and update one or more of the anomaly detection models over time based on the received feedback.

In some implementations, the historical clinical data messages and the new clinical data messages comprise messages that were generated by the same set of clinical information resources associated with the same hospital system. With these implementations, the anomaly detection models may be used to monitor and detect data discrepancies associated with the same integration project over time. In other implementations, the historical clinical data messages can comprise message that were generated by one or more first clinical information resources associated with a first same hospital system and the new clinical data messages can comprise messages that were generated by one or more second clinical information resources associated with a second same hospital system. With these implementations, the mapping logic configured for a previous hospital system can be used as a starting point for the integration project for a new hospital system, and the anomaly detection models can be used to identify discrepancies between the mapping logic for the previous system and the new system for adapting to the new system.

In some embodiments, elements described in connection with the disclosed systems can be embodied in different forms such as a computer-implemented method, a computer program product, or another form.

### DESCRIPTION OF THE DRAWINGS

FIG. 1 illustrates a block diagram of an example, non-limiting system that facilitates detecting data discrepancies associated with clinical data integration in accordance with one or more embodiments of the disclosed subject matter.

FIG. 2 presents an example FHIR bundle and an example of how one FHIR path/key may be encoded in accordance with one or more embodiments of the disclosed subject matter.

FIG. 3 illustrates a flow diagram of an example process for generating anomaly detection models adapted to detect data discrepancies associated with clinical data integration in accordance with one or more embodiments of the disclosed subject matter.

FIG. 4 illustrates a flow diagram of an example process for employing anomaly detection models to detect data discrepancies associated with clinical data integration in accordance with one or more embodiments of the disclosed subject matter.

FIG. 5 illustrates another example, non-limiting system that facilitates detecting data discrepancies associated with clinical data integration in accordance with one or more embodiments of the disclosed subject matter.

FIG. 6 illustrates another example, non-limiting system that facilitates detecting data discrepancies associated with clinical data integration in accordance with one or more embodiments of the disclosed subject matter.

FIGS. 7A-7B present an example graphical user interface that facilitates reviewing integration report in accordance with one or more embodiments of the disclosed subject matter.

FIG. 8 illustrates a high-level flow diagram of an example process for detecting data discrepancies associated with clinical data integration in accordance with one or more embodiments of the disclosed subject matter.

FIG. 9 illustrates a high-level flow diagram of another example process for detecting data discrepancies associated with clinical data integration in accordance with one or more embodiments of the disclosed subject matter.

FIG. 10 illustrates a block diagram of an example, non-limiting operating environment in which one or more embodiments described herein can be facilitated.

FIG. 11 illustrates a block diagram of another example, non-limiting operating environment in which one or more embodiments described herein can be facilitated.

### DETAILED DESCRIPTION

The following detailed description is merely illustrative and is not intended to limit embodiments and/or application or uses of embodiments. Furthermore, there is no intention to be bound by any expressed or implied information presented in the preceding Background section, Summary section or in the Detailed Description section.

As discussed above, the medical data integration processes is a time consuming and error prone process that requires the expertise of integration engineers to manually develop complex mapping logic for mapping clinical data exported from various disparate clinical information systems in one or more native formats into a single canonical target format. With this problem in mind, the disclosed subject matter provides automated tools for detecting integration errors associated with an integration project and notifying integration engineers regarding the detected integration engineers so that they may be properly remediated. To facilitate this end, the disclosed techniques formulate detecting mapping errors resulting from the mapping logic as an anomaly detection problem and uses machine learning techniques to solve the anomaly detection problem.

In one or more embodiments, the disclosed techniques break the target data format into a defined set of data description paths, wherein each data description path is configured to include a defined set of data elements. For example, each data description path can be defined by one or more data fields corresponding to defined data elements. In this regard, each data description path can be correlated to a different data channel, wherein the type of data and the characteristics of the data associated with each data channel is different. The disclosed techniques further employ historical mapped clinical data in the target format from a previous successful integration project as training data to develop separate anomaly detection models for each data description path. In particular, the historical mapped clinical data can comprise clinical data for a hospital system in the target format that was generated by multiple different clinical information systems associated with the hospital system in one or more native formats and mapped via a previously configured mapping function into the target format. The disclosed techniques assume the previously configured mapping function to be error free (or sufficiently error free) and thus assume the historical mapped data to represent correctly mapped data elements and characteristics of those data elements for each of the defined data description paths. In this regard, in accordance with the multiple data channel analogy, the historical mapped data is assumed to provide, for each data channel, a representation of the correct types of data elements to be included in each data channel and the correct values for those data elements.

The disclosed techniques further employ machine learning to learn the normal/correct distributions of the types of data elements and values for those data elements for each of the different data description paths (i.e., data channels). In various embodiments, this can involve training separate deep learning models for each of the data description paths to learn the normal distributions of the types of data elements and values for those data elements, and further configuring the deep learning models to estimate the likelihood that a new set of mapped data for each data description path is normal, that is, the specific types of data elements and/or the characteristics of the data elements (e.g., values) is normal. Once trained, these anomaly detection models can be employed to evaluate the accuracy of newly mapped clinical data mapped via the same mapping function for a new integration project for a new hospital system. The anomaly detection models can also be employed to continuously monitor the conversion accuracy of mapped clinical data for the original system from which the training data was collected to detect newly arising integration errors over time.

In this regard, one or more embodiments of the disclosed subject matter provide systems, computer-implemented methods, apparatus and/or computer program products that provide tools for loading or otherwise receiving a corpus of mapped historical clinical data for a previously successful clinical data integration project for a hospital system (or a similar system) that was mapped into a target format via a previously configured mapping function. The historical mapped clinical data can represent a collection of clinical data generated by one or more clinical information systems over a past period of time that is sufficient to provide a representative distribution of the different types of clinical data messages and content of the clinical data messages that the one or more clinical information systems produce over time. For example, the historical mapped clinical data may include aggregated clinical data generated over a past week, month or more. In most scenarios, the clinical information systems involved in the integration project will include a plurality of disparate clinical information systems that provide a wide range of different types of clinical data in one or more different native formats. The specific clinical information systems that provide the historical clinical information is known at the start of the integration project and used by the integration engineers to develop the predefined mapping logic.

The disclosed systems further include machine learning training logic for training the anomaly detection models using the historical mapped clinical data and runtime logic for applying the anomaly detection models to newly mapped clinical data mapped using the same previously defined mapping logic (or in some implementations a different mapping logic) to detect mapping errors. As noted above, the newly mapped clinical data can include data generated by the same clinical information systems that generated the historical clinical data and/or a different set of clinical information systems associated with a new medical data integration project. The disclosed systems further provide tools for loading or otherwise receiving the newly mapped clinical data as corpus of mapped clinical data generated by the clinical information systems aggregated over a past period of time and/or in real-time.

The disclosed systems further provide logic for generating integration report data regarding the results of the anomaly detection models and providing the integration report data to integration engineers to facilitate reviewing detected mapping errors. For example, the disclosed systems can generate the integration report data in human interpretable format that can be rendered via a suitable display device. In some embodiments, the integration report data can include a list of all the defined data description paths and anomaly scores determined for each of the defined data description paths that indicate a measure of the amount and/or severity of the detected mapping errors associated with each of the data description paths. The integration report data can also include representative data samples for each of the defined data description paths associated with anomaly scores that exceed a maximum anomaly score threshold and thus are considered to be likely to include an unacceptable level of mapping errors. In some embodiments, the disclosed systems can provide an interactive graphical user interface and corresponding interaction logic that provides for interacting with the integration report data to facilitate evaluating the representative data samples to gain a better understanding of the potential mapping errors and performing root cause analysis to determine potential causes of the mapping errors. The interactive graphical user interface can further provide a mechanism for receiving user feedback regarding the accuracy of the anomaly scores as determined based on manual review of the representative data samples. For example, after investigating representative data samples for a data description path that received a significantly high anomaly score, the integration engineer may decide that the data samples were actually mapped correctly and thus provide feedback indicating the anomaly score for that data description path is not accurate. The disclosed systems further provide a continuous learning regime in which the received feedback is used by the machine learning logic to retrain and update (e.g., fine tune) the corresponding anomaly detection models over time.

Various embodiments of the disclosed subject matter are directed to medical data integration and detecting mapping errors associated with mapping clinical data from one or more native formats into a single canonical target format. However, the disclosed techniques can be extended to other domains for detecting mapping errors associated with other types of data. In this regard, the term “clinical data” is used herein to referred to any type of information associated with a healthcare system ranging from patient related information (e.g., including determinants of health and measures of health and health status to documentation of care delivery) to operational and administrative information. Different types of clinical data are captured for a variety of purposes and stored in numerous databases across healthcare systems and/or reported in real-time over the course of operation of the healthcare system for usage by clinicians and/or consumption by clinical applications. Some example types of clinical data that may be included in the mapped clinical data evaluated by the disclosed systems can include (but is not limited to): patient electronic health record (EHR) data, patient care progression data, patient physiological data, patient medical image data and associated metadata (e.g., acquisition parameters), radiology report data, clinical laboratory data, medication data, medical procedure data, pathology report data, hospital admission data, discharge and transfer data, discharge summary data, progress note data, medical equipment and supplies data, hospital administration data, hospital operational data, patient scheduling data, financial/billing data, and medical insurance claim data.

The terms “algorithm” and “model” are used herein interchangeably unless context warrants particular distinction amongst the terms. The terms “AI model” and “ML model” are used herein interchangeably unless context warrants particular distinction amongst the terms.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

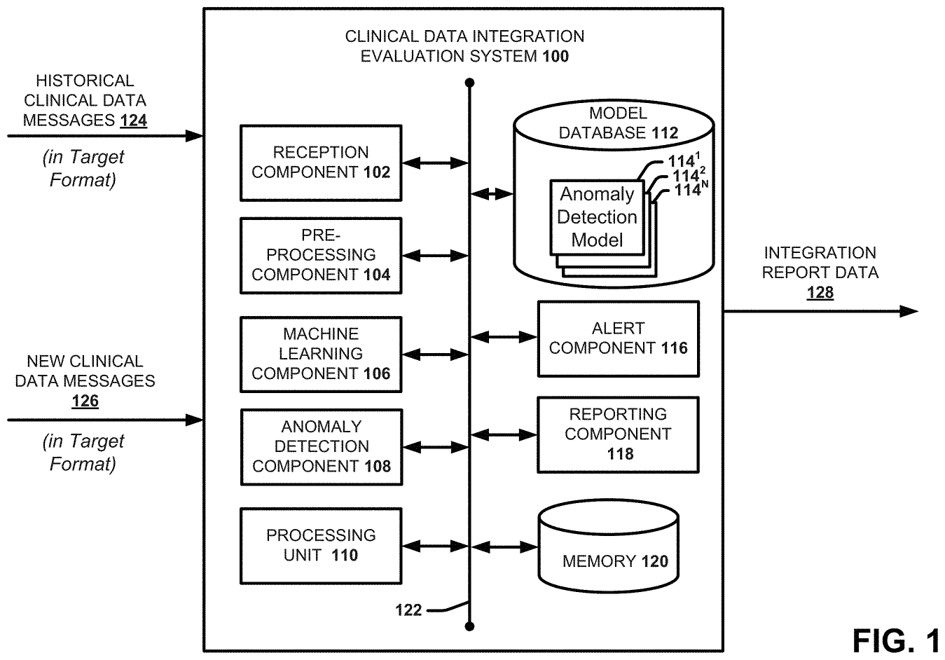

FIG. 1 illustrates a block diagram of an example, non-limiting clinical data integration evaluation system 100 (also referred to as system 100) that facilitates detecting data discrepancies associated with clinical data integration in accordance with one or more embodiments of the disclosed subject matter. Embodiments of systems described herein can include one or more machine-executable components embodied within one or more machines (e.g., embodied in one or more computer-readable storage media associated with one or more machines). Such components, when executed by the one or more machines (e.g., processors, computers, computing devices, virtual machines, etc.) can cause the one or more machines to perform the operations described.

In this regard, system 100 includes reception component 102, pre-processing component 104, machine learning component 106, anomaly detection component 108, alert component 116, and reporting component 118, all of which can be or include machine-executable components embodied within one or more machines (e.g., embodied in one or more computer-readable storage media associated with one or more machines), which when executed by the one or more machines (e.g., processors, computers, computing devices, virtual machines, etc.) can cause the one or more machines to perform the operations described. System 100 further includes a model database 112 that can include a plurality of anomaly detection models, respectively identified anomaly detection models 114^{1-N}. As described infra, these anomaly detection models 114^{1-N} can respectively correspond to computer executable models or algorithms adapted estimate the likelihood that the data mapped to respective defined data description path of a target data format is correctly mapped. A separate anomaly detection model 114^{1-N} model can be generated for each of the defined data description paths. In this regard, the clinical data integration evaluation system 100 can be any suitable machine that can execute one or more of the operations described with reference to the reception component 102, pre-processing component 104, the machine learning component 106, the anomaly detection component 108, the alert component 116, the reporting component 118, the anomaly detection models 114^{1-N}, and other components described herein.

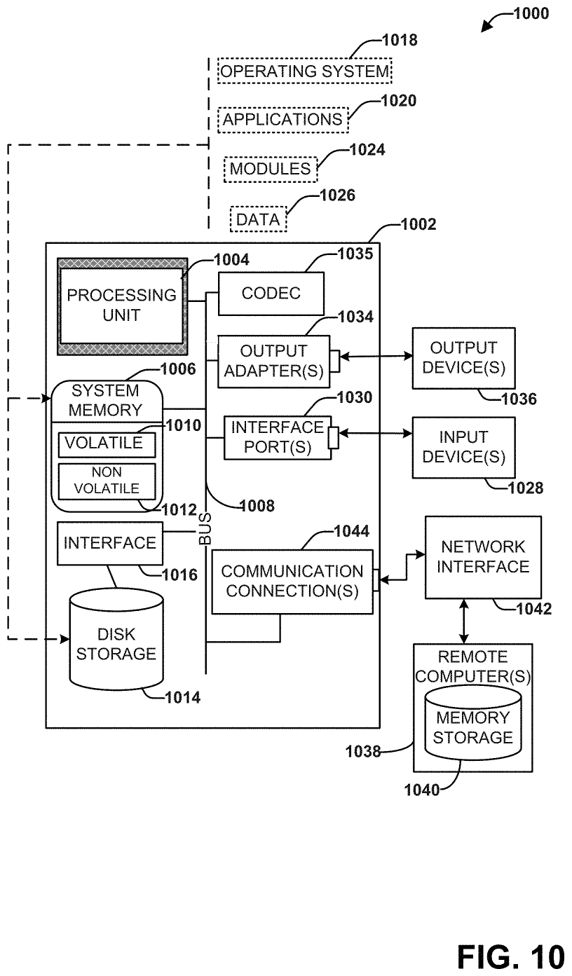

As used herein, the machine can be and/or can include one or more of a computing device, a general-purpose computer, a special-purpose computer, a quantum computing device (e.g., a quantum computer), a tablet computing device, a handheld device, a server class computing machine and/or database, a laptop computer, a notebook computer, a desktop computer, a cell phone, a smart phone, a consumer appliance and/or instrumentation, an industrial and/or commercial device, a digital assistant, a multimedia Internet-enabled phone and/or another type of device. System 100 can also be or correspond to one or more real or virtual (e.g., cloud-based) computing devices. System 100 can further include or be operatively coupled to a least one memory 120 that stores the computer executable components (e.g., the reception component 102, the pre-processing component 104, the machine learning component 106, the anomaly detection component 108, the alert component 116, the reporting component 118, the anomaly detection models 114^{1-N}, and other components described herein). The memory 120 can also store any information received by the system 100 (e.g., the historical clinical data messages 124 and the new clinical data messages 126) and/or generated by the system 100 (e.g., integration report data 128). System 100 can further include or be operatively coupled to at least one processing unit 110 (or processor) that executes the computer-executable components stored in the memory 120, and a system bus 122 that communicatively couples the respective components of the system 100 to one another. Examples of said and memory 120 and processing unit 110 as well as other suitable computer or computing-based elements, can be found with reference to FIG. 10 (e.g., with reference to processing unit 1004 and system memory 1006), and can be used in connection with implementing one or more of the systems or components shown and described in connection with FIG. 1 or other figures disclosed herein.

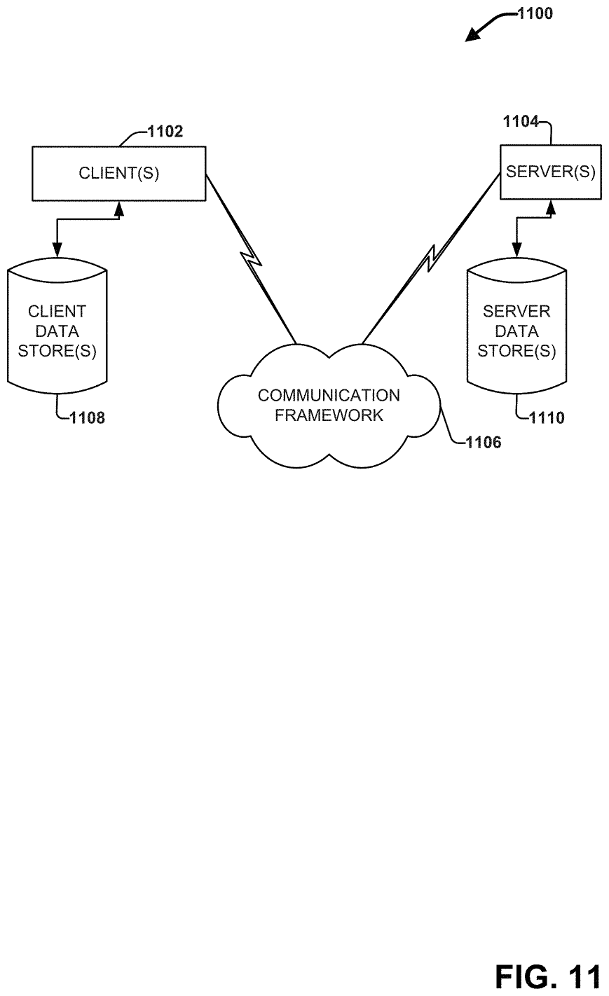

The deployment architecture of system 100 can vary. In some embodiments, system 100 can be deployed a local computing device. In other embodiments, one or more of the components of system 100 can be deployed in a cloud architecture, a virtualized enterprise architecture, or an enterprise architecture wherein one the front-end components and the back-end components are distributed in a client/server relationship. With these embodiments, the features and functionalities of one or more of the reception component 102, the pre-processing component 104, the machine learning component 106, the anomaly detection component 108, the alert component 116, the reporting component 118, the anomaly detection models 114^{1-N}, the processing unit 110 and the memory 120 (and other components described herein), can be deployed as a web-application, a cloud-application, a thin client application, a thick client application, a native client application, a hybrid client application, or the like. Various example deployment architectures for system 100 (and other systems described herein) are described infra with reference to FIGS. 10-11.

The clinical event data integration system 100 is configured to analyze clinical data mapped from one or more native formats into a target format via defined mapping logic to detect mapping errors and generate integration report data 128 regarding any detected mapping errors. In this regard, system 100 is designed to evaluate integration errors associated with a clinical data integration project for a hospital system (or a similar system) in which clinical data is exported from one or more clinical information systems in one or more native formats and mapped to a single canonical target format via a defined mapping function. The defined mapping function employs defined mapping logic in the form of mapping rules, translation tables and/or associated scripts to convert data messages in the native format or formats into the target format. As noted above, these mapping rules are manually defined by integration engineers and are tailored for each specific integration project.

In the context of medical data integration, the process for defining and maintaining these mapping rules becomes significantly complex. In particular, the medical data integration process typically involves converting a variety of types of clinical data exported from a plurality of disparate clinical information systems. In these environments, many of the clinical information systems were never designed to easily export data to external consumers. For example, some information systems may export data in different versions of the HL7™ format, while others may use proprietary file formats, flat files or external databases to export data. For instance, different electronic medical record (EMR) systems may use different data formats for defining patient data. In addition, there are tens of thousands (or more) of different medical terms and different clinical information systems may use different medical terminology and corresponding code sets for describing clinical data items. The mapping function must be able to recognize all medical terms and codes used by the different clinical information systems and translate them to the proper terminology and data fields in accordance with the target data description format. For example, one mapping rule may instruct the conversion engine to take a specific medical term in data field 8 of one data string in the native format and convert it to a corresponding medical code used in the target format and apply it to data field 5 of different data string in the target format. Such rules may need to be defined for all potential data fields and data elements associated with the native format and the target format. Further, many clinical data items are unique by design and thus difficult to recognize and translate, such as unique patient identifiers, specific site information (e.g., time, data, city, state, etc.), floating point values with high precision, semi-structured text fields, and free text fields. Accordingly, defining the mapping rules for a medical data integration project can be an extremely tedious process that may take several weeks or months to complete and validate or debug.

With this problem in mind, the clinical data integration system 100 provides automated tools for detecting mapping errors attributed to previously defined mapping rules developed for a medical data integration project. To facilitate this end, the clinical data integration evaluation system 100 breaks the target data format into a defined set of data description paths, wherein each data description path is configured to include a defined set of data elements. For example, each data description path can be defined by one or more data fields corresponding to defined data elements. In this regard, each data description path can be correlated to a different data channel, wherein the type of data and the characteristics of the data associated with each data channel is different. In other words, the mapped clinical data can be broken into multiple different communication channels and each channel should hold only data of specific types (e.g., values, units of measure, unique patient identifiers, etc.). Information defining the target data format and the defined data description paths (i.e., data channels) can be stored in memory 120 and/or another suitable memory structure accessible to the clinical data integration system 100. In various embodiments, each of the defined data description paths can be assigned a unique path identifier. The information defining the different data description paths can vary based on the data description model of the target data file format used, which can vary. In general, the information defining the different data description paths can include the one or more data fields included in each data description path, the syntax of the one or more data fields, and the name of the data element or data object corresponding to each data field. In some implementations, the information defining the different data path descriptions can also define the type (e.g., a class type) of the data element or data object corresponding to each field and/or a description of the characteristics of the data elements or data objects that are valid for each data field. As described in greater detail below, in some implementations, this data element type information and/or characteristic information can be learned by the machine learning component 106 during training of the anomaly detection models.

In one or more embodiments, the target data format comprises the Fast Healthcare Interoperability Resources (FHIR™). The FHIR data model is a standard for defining clinical content and other pertinent system information such as the EHRs capabilities and hospital administrative information in a consistent, structured yet flexible modular format. FHIR data is intended to be consumed by computers and is thus written in a computer-interpretable format, but it is structured in a way that allows for human readability. In FHIR, health care data is broken down into categories such as patients, laboratory results, and insurance claims, among many others. Each of these categories is represented by a FHIR resource, which defines the component data elements, constraints on data, and data relationships that together make up an exchangeable patient record. Each resource contains data elements necessary for its specific use cases and links to relevant information in other resources. For example, the patient resource contains basic patient demographics, contact information, and links to a clinician or organization stored in a different resource. Because they are based on modern World Wide Web technologies, resources use Uniform Resource Locators, or URLs (also generally known as web addresses), to be located within a FHIR system implementation. The FHIR data model is built from a set of modular components called resources. Resources have a common definition and method of representation, a common set of metadata, and a human-readable part. FHIR Resources have strict restrictions on intermixing of values with differing data types, like strings and numeric values. The FHIR data model is designed specifically for the web applications and provides resources built on the XML, JSON, HTTP, Atom, and OAuth data format structures. These data structures (e.g., XML, JSON, etc.) use a hierarchical tree structure to describe distinct data messages organized per FHIR resource.

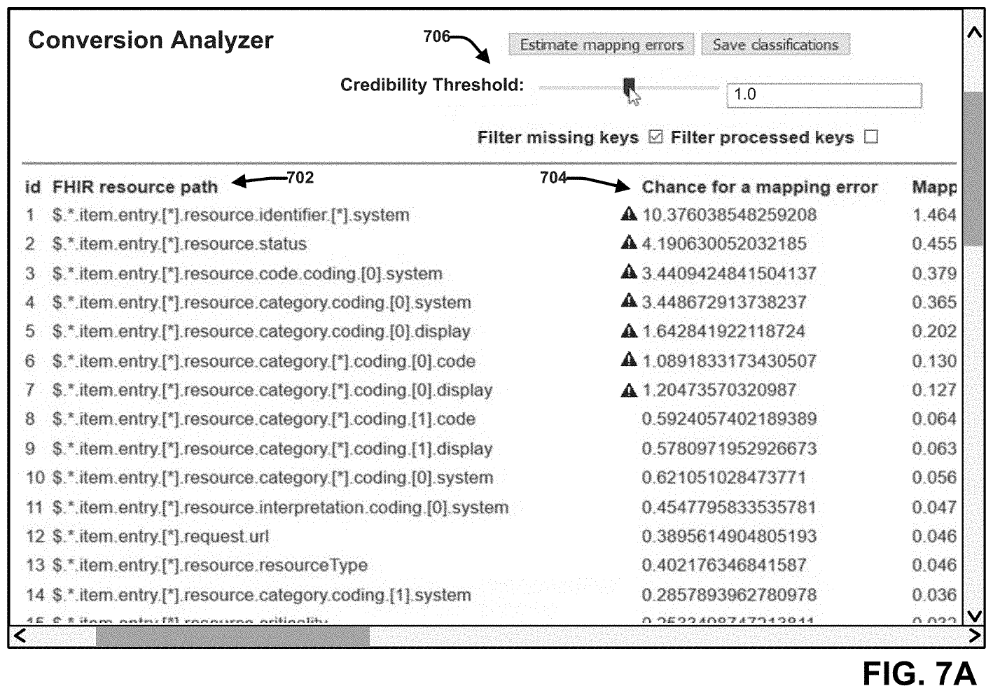

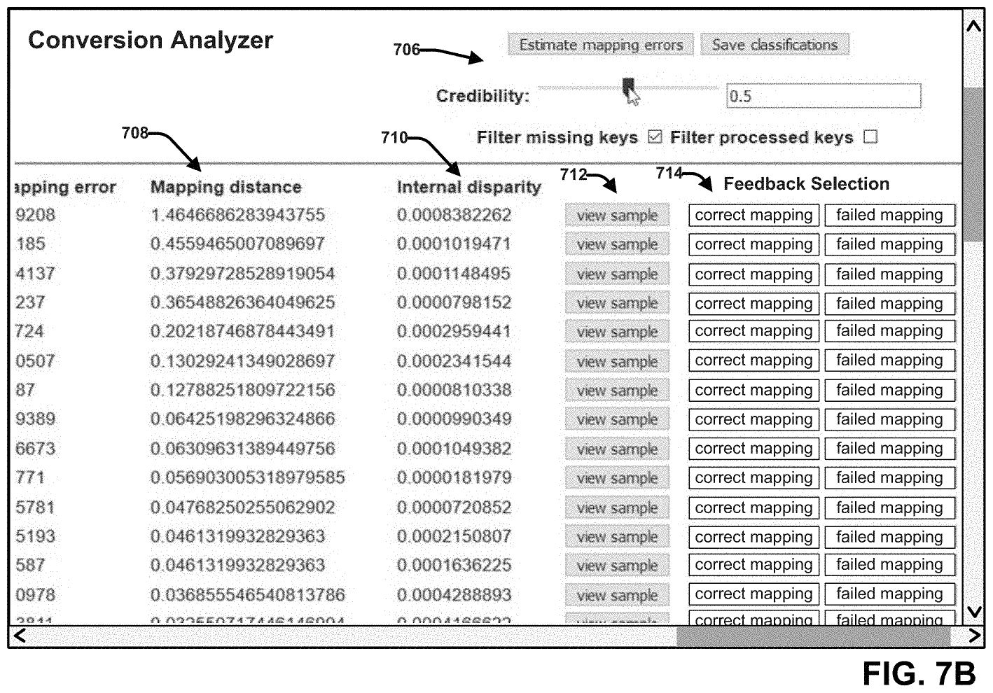

In some embodiments in which the target data format is FHIR, the different data description paths can correspond to distinct FHIR resource path (also referred to herein as an FHIR key), wherein each FHIR resource path corresponds to a distinct JSON path. In this regard, the term data description path as applied to FHIR is used to reflect the different paths in a FHIR resource, wherein FHIR is characterized by the fact that each such path carries its semantic context. In particular, assuming a collection of data messages in one or more native formats are converted via the mapping function into FHIR messages, each FHIR message can be represented as JSON object that has a hierarchical tree structure and contains multiple FHIR resource paths. The hierarchical tree structure can be flattened so that the path from the root of each JSON object is used as the FHIR resource path for each associated data field having a value (e.g., a numerical measurement value, a unique identifier or medical code for a particular drug, procedure, etc.). For example, assuming one data field corresponds to a recorded systolic blood pressure value for a patient, the value entered for that data field should include an acceptable number for a systolic blood pressure measurement (e.g., ranging between 100 millimeters of mercury (mmHg) and 200 mmHg for systolic). By using the JSON path syntax, each path in a JSON archive can be uniquely indexed as unique FHIR resource paths. Two example FHIR resource paths generated in this manner are listed as items 1 and 2 below with some additional examples illustrated in FIGS. 7A-7B.

1. $.*.item.entry.[*].resource.generalPractitioner.[0].reference

2. $.*.item.entry.[*].resource.category.[*].coding.[0].code

As illustrated in the above two examples, each distinct FHIR resource path contains a defined set of data fields, arranged according to a defined syntax, wherein each data field corresponds to a specific type of data element, wherein at least one of the data elements corresponds to value. In the examples above, the value data fields are represented by the text fields. The value data fields in the above examples comprise placeholder terms that represent the type or class of data item to be included in the corresponding data field. These value data fields are not filled in with actual values because these FHIR resource paths correspond to the definitions of two example resource paths. In practice, each time a native clinical data message is converted into an FHIR message by the defined mapping function, the value data fields should include a corresponding value (e.g., a numerical value, a unique medical code or term, a unique identifier, etc.).

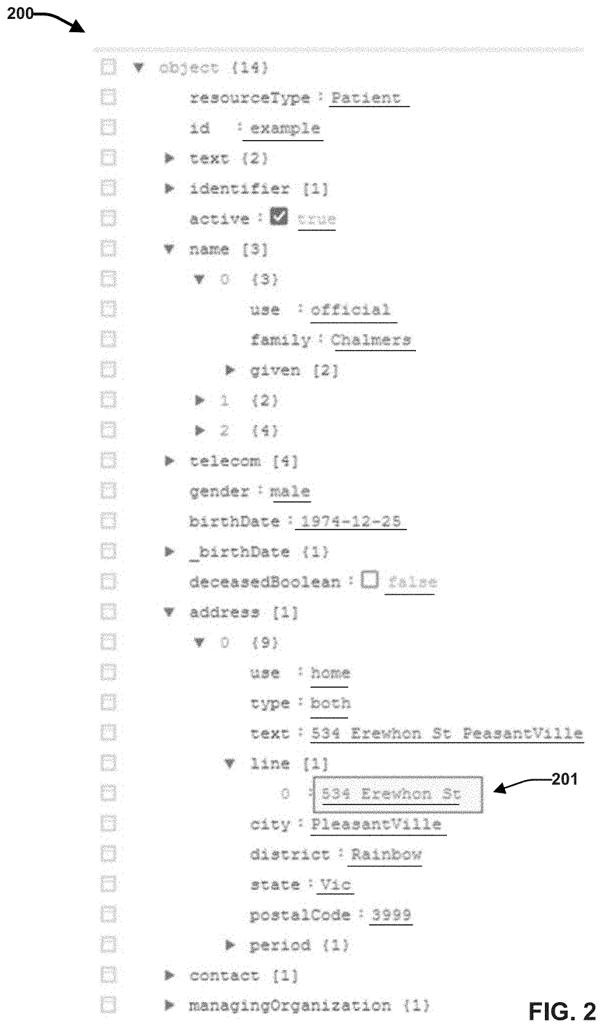

FIG. 2 presents an example FHIR bundle 200 in accordance with one or more embodiments of the disclosed subject matter. In various embodiments, the FHIR bundle corresponds to an FHIR clinical message or FHIR object (referred to in the FHIR bundle 200 as object (14)) and includes a plurality value data fields whose corresponding values are underlined. In various embodiments, a separate FHIR path/key can be defined for each value data field. For instance, in one example as applied to the value in data field 201, the FHIR path or key for “534 Erewhon St.” can be represented with the following unique coding: *.address.[0].line.[0], wherein the type of this value data field correspond to an address. In this regard, FHIR resources are sent in the form of FHIR bundles. Each bundle includes one or more FHIR resources. Each resource is represented using a transfer format (e.g., JSON). In each resource there is a single value for each key. This s a 1:1 mapping. When we consider the entirety of all received FHIR bundles and collect their values into sets each key can be thought of as indexing its set of received values. In this context the mapping can be considered 1:n. In this regard, if an FHIR key such refers to a single FHIR resource, they will index a single value, yet if they relate to all (part) of received bundles they are more likely to index a set of values for different value fields. Thus, in some implementations, each FHIR path/key can be defined by a set (wherein the set includes two or more) of different value fields while when the refer to two or more different FHIR resources. In other implementations, each FHIR path/key can be defined by a single value field when the refer to a single FHIR resource.

With reference to again to FIG. 1, by breaking the mapped clinical data messages in the target format down in into a defined set of possible data description paths (e.g., FHIR resource paths such as those illustrated above or similar data description paths), the clinical data integration evaluation system 100 can formulate the mapping error detection problem as a function of detecting erroneous values in the value data fields for each of the defined data description paths. In this regard, the clinical data integration evaluation system 100 assumes a mapping error is expected to manifest itself as a different set of values with respect to the set of values each data description path (e.g., each FHIR resource path) had witnessed in previous installations. Although reference to a “set” of values is used, it should be appreciated that the set may include one or more values. In this regard, some data description paths may include only a single data field corresponding to a value data field. The clinical data integration evaluation system 100 further assumes that over a sufficiently long duration of collection of mapped data messages for a previously successful integration project (e.g., assuming the mapping function is error free or sufficiently error free), a distribution of all or most attainable valid values per each defined data description path will be encountered.

Based on this framework, the machine learning component 106 employs historical mapped data in the target format from a previous successful integration project as training data to develop separate anomaly detection models 114^{1-N} for each data description path (e.g., each FHIR resource path or a similar data description path). In particular, the historical mapped data can comprise clinical data for a hospital system (or a similar system) in the target format that was generated by one or more clinical information systems associated with the hospital system in one or more native formats and mapped via a previously configured mapping function into the target format. The disclosed techniques assume the previous integration project to be successful, meaning that the previously configured mapping function is assumed to be error free (or sufficiently error free), and thus assume the historical mapped data to represent correctly mapped data elements and characteristics of those data elements (e.g., values) for each of the defined data description paths. In this regard, in accordance with the multiple data channel analogy, the historical mapped data is assumed to provide, for each data channel, a representation of the correct types of data elements to be included in each data channel and the correct values for those data elements.

In the embodiment shown in system 100, this historical mapped data is represented by the historical clinical data messages 124. For example, in implementations in which the target format comprises FHIR, the historical clinical data messages 124 can comprises a collection of FHIR messages. In this regard, the historical clinical data messages 124 represent a collection of clinical data generated by one or more clinical information systems over a past period of time that is sufficient to provide a representative distribution of the different types of clinical data messages and content of the clinical data messages that the one or more clinical information systems produce over time. For example, the historical clinical data messages 124 may include aggregated clinical data generated by the one or more clinical information systems over a past week, month or more. In most scenarios, the clinical information systems involved in the integration project will include a plurality of disparate clinical information systems that provide a wide range of different types of clinical data in one or more different native formats. The specific clinical information systems that provide the historical clinical information is known at the start of the integration project and used by the integration engineers to develop the predefined mapping logic.

The reception component 102 thus receives the historical clinical data messages 124 in the target format (e.g., FHIR or another target format). The manner in which the reception component receives the historical clinical data messages 124 can vary. In some embodiments, the reception component can provide a loading function for loading (e.g., downloading) the historical clinical data messages 124 in a batch loading procedure from another data storage system of device where the historical clinical data messages are aggregated and stored. In other embodiments, the reception component 102 can receive and aggregate the historical clinical data messages 124 over time from the one or more clinical information systems following conversion by the conversion engine that implements the mapping function. The conversion engine may be executed by one or more external systems or devices.

The pre-processing component 104 can pre-process the historical clinical messages to prepare them for further processing (e.g., by the machine learning component 106). In particular, the pre-processing component 104 can index the historical clinical data messages 124 based on the defined data description paths to generate an indexed group of data samples for each of the defined data description paths. For example, in some embodiments in which the target data format comprises the FHIR format (or a similar format employing the JSON structure or a similar data representation structure) and each of the historical clinical data messages are represented as JSON objects (or similar data objects), the pre-preprocessing component 104 can segment each historical clinical data message into its corresponding FHIR data paths (or FHIR keys). In this regard, the pre-processing component 104 can flatten each hierarchical JSON object corresponding to each historical clinical data message into separate JSON paths from the root of each JSON object, wherein each separate JSON path corresponds to a specific FHIR data path (or FHIR key). The pre-processing component 104 can further groups the data paths belonging to a same key together to generate separate groups of data samples for each of the FHIR keys. In this regard, each of the data samples belonging to a same data description path (e.g., FHIR data path or FHIR key) comprises a distinct set of data elements included in defined data fields in the form of a data string (e.g., corresponding to the FHIR data path examples described above), wherein at least some of the data fields comprise values.

The collection of data samples associated with each data description path (e.g., each FHIR path/key) computed for the historical clinical data messages 124 are assumed to provide a representative distribution of the valid or normal set of values for the corresponding data description path. The clinical data integration evaluation system 100 is designed on the technical assumption that native format to target format mapping failures are likely to manifest themselves as larges differences between the value distribution of the validated mapping determined for the training set and newly mapped data for a new hospital system/integration project or the previous system providing the training data (e.g., following system updates and/or changes). In the embodiment shown, this newly mapped data is represented by the new clinical data messages 126. In this regard, the new clinical data messages 126 can correspond to a new collection of clinical data messages converted from one or more native formats into the target data format via the same mapping function used to convert the historical clinical data messages 124. In some embodiments, the mapping function used to generate the new clinical data messages however may be different.

One way the clinical data integration evaluation system 100 can utilize this insight is to compute the value distribution for each data description path (e.g., each FHIR path/key), for both the training set (i.e., the historical clinical data messages 124) and the new data (i.e., the new clinical data messages 126), measure the distribution distance using (e.g., using a Bhattacharyya distance metric or another distance metric) and determine an anomaly score as a function of the distribution distance, wherein the greater the distance, the higher the anomaly score. With these embodiments, the machine learning component 106 can compute the value distributions for each data description path based on the pre-processed training data. For instance, assume one of the data description paths correspond to the FHIR path of example 1 above (e.g., $.*.item.entry.[*].resource.category.[*].coding.[0].code) which includes three different value fields. The training data set for this data description path will include a collection of data samples (e.g., data strings) that correspond to this FHIR path, wherein each of the data samples will include three mapped values for these value fields. In this regard, each data sample comprises a set of three different values for the three different value fields. These sets of values are assumed to provide a normal distribution of the valid/correct values that this FHIR data path should include for each of the three value fields, as the training data is assumed to be correctly mapped via the mapping function (e.g., the mapping function is assumed to be error free or substantially error free at the time the training data is generated). According to this example, the machine learning component 106 can determine the value distribution for this FHIR as a function of the distribution of all the values in each of the three value data fields for each of the data samples. The machine learning component 106 can similarly compute the value distributions for all of the defined data description paths.

The anomaly detection component 108 can further compute the value distributions for each of the data description paths for the new clinical data messages 126 in the same manner. The pre-processing component 104 can also pre-process the new clinical data messages 126 in the same manner described above with respect to the historical clinical data messages 124 (i.e., the training data) to generate a collection of data samples for each data description to path prepare the new clinical data messages 126 for computing the value distributions for each of the data description paths. In some embodiments, the machine learning component 106 and the anomaly detection component 108 can employ a histogram of raw string values to model and compute the value distributions for each of the defined data description paths (e.g., each FHIR path/key). With these embodiments, all strings can be lowercased, and extraneous whitespaces can be discarded.

For each of the data description paths (e.g., each FHIR path/key), the anomaly detection component 108 can further compare the value distributions of the training data set with the value distributions of the new clinical data messages 126, determine the distribution distance between the training value distributions and the new clinical data message value distributions, and determine an anomaly score for each of the data description paths based on distribution distances. In this regard, data fields with low internal distance are considered more credible when compared to the testing set. The metric for the anomaly scores can correspond to the distribution difference values or another metric that reflects the degree/amount of the distribution difference values.

To judge if a distribution difference-based anomaly score might hint at conversion error, the anomaly detection component 108 can further compare the anomaly scores determined for each of the data description paths based on the new clinical data messages to a maximum anomaly score threshold to identify data description paths associated with potential mapping errors as those with anomaly detection scores exceeding the threshold. The maximum anomaly score for each data description path can vary or be the same and manually configured to a desired threshold value. Additionally, or alternatively, the machine learning component 106 can determine the maximum anomaly score thresholds for each of the data description paths. To facilitate this end, the machine learning component 106 can determine a baseline to the internal variability of the distance between the values in each data description path (e.g., each FHIR resource path/key) based on the training data (i.e., the historical clinical data messages 124). The machine learning component 106 can further determine the maximum anomaly scores for each data description path (e.g., FHIR resource path/key) based on the baseline variability distances determined for each of the data description paths. For example, the machine learning component 106 can set the maximum anomaly scores to be equal to the baseline variability distances or set the maximum anomaly scores to be a defined amount greater than the baseline variability distances. To facilitate this end, for each data description path, the machine learning component 106 can split the distribution difference scores of random splits of the training set and average the results to yield a baseline distribution difference for each of the data description paths. With these embodiments, the maximum anomaly score for each data description path can vary or be set to the same value as function of the average of the baseline variability distance for all the data description paths.

Additionally, or alternatively, the machine learning component 106 can employ machine learning to learn the normal/valid distributions of the values for each of the different data description paths (e.g., FHIR paths/keys). In this regard, a histogram representation of the value distributions for the value fields in a data description path serves well when the raw string values for value fields include a small discreet set of values. For instance, one example data field that satisfies this criterion could correspond to a data filed for a diagnosis result which can either be one of two values, positive or negative. However, when the values for the data field or fields in a data description path (e.g., an FHIR path/key) represent a large set of discreet values or for other data types, a histogram representation of the value distribution may be less useful. For example, data fields that may include a large set of discrete values could include but are not limited to:

1. Data fields representing any type of random identifier, such as unique patient identifiers (e.g., patient names or anonymized patient identifiers in the form of a global unique identifier (GUID), as these values may be designed to be unique hence induce disjoint sets of values resulting in infinite distance of their probability distribution which signify a mapping failure);

2. Data fields that reflect a specific clinical information system that is unique to the training data set (e.g., that would always be different or missing in the new clinical data messages 126);

3. Data fields that reflect transitory concepts such as times, dates, locations (e.g., state, city);

4. Data fields with continuous values (e.g., floating values or integer values with a large dynamic range and a wide value distribution);

5. Free text data fields; and

6. Hierarchical fields where part of the field represent a stable concept while other parts are transient.

To handle these types of fields, in some embodiments, the machine learning component 106 utilize the semantics of each field to handle them accordingly. In particular, the machine learning component 106 can employ one or more machine learning techniques to identify data fields included in the training data that correspond to any of the data fields corresponding to types 1-6 above and/or otherwise comprise a large set of discreet values (e.g., with respect to a measurable threshold). The machine learning component 106 can further classify each of these data fields with a defined class type and define the valid characteristics of the values of the data fields for each class type based on the learned characteristics of the values associated with each class type (as learned based on analysis of the historical clinical data messages 124). Information defining these data fields for each of the data description paths and the valid characteristics of these data fields can further be stored in memory 120 and employed by the anomaly detection component 108 to determine anomaly scores for each of the data fields. In this regard, the anomaly detection component 108 can evaluate the data fields for each class type in the new clinical data messages 126 using specialized handling based on the defined valid characteristics of the values for the corresponding data fields to estimate anomaly scores for the corresponding data fields based on the values mapped to those data fields in the new clinical data messages 126. For example, in some implementations, for data fields having a class type corresponding to types 1-3 above, the anomaly detection component 108 can determine whether the values have the characteristics defined for the corresponding class type, wherein the valid characteristics were previously learned and defined by the machine learning component 106. For example, as applied to a date data field, the machine learning component 106 can define any value for the date data field that corresponds to a date in one or more formats (e.g., Nov. 11, 2020, Nov. 11, 2020, 11/20/2020, 11.20.2020, etc.) as being valid, otherwise invalid. With these implementations, the anomaly detection component 106 can determine an anomaly score for these data fields based on the number of valid and invalid values detected. In implementations in which the data fields correspond to type 4 above, the machine learning component 106 and the anomaly detection component 108 can respectively determine the distributions of the corresponding values for the training data and the new clinical data using a mixture of gaussian distributions. The anomaly detection component 108 can further determine anomaly scores for those data fields in the new clinical data 108 based a measure of difference between the respective Gaussian distributions for the training data and the new clinical data.

Additionally, or alternatively, the machine learning component 106 can train separate anomaly detection models 114^{1-N} for each of the data description paths to learn the normal distributions of these values for the corresponding data fields, and further configure the anomaly detection models 114^{1-N} to estimate the likelihood that the set of values mapped data for each data description path for the new clinical data messages 126 is normal, that is, the new set of values falls within an acceptable range of deviation from the training set. For example, in some embodiments, the machine learning component 106 can configure the anomaly detection models 114^{1-N} to generate anomaly scores for each of the data description paths based on the new clinical data messages 126, wherein the anomaly scores reflect a measure of the distribution differences between the values of the new clinical data messages and the values of historical clinical data messages 124. In this regard, the higher the anomaly score, the higher the likelihood that the corresponding data description path is associated with mapping errors. With these embodiments, the anomaly detection component 108 further employ a maximum anomaly score (or scores) for each data description path to identify description paths associated with potential mapping errors as those with anomaly detection scores exceeding the maximum anomaly score. The maximum anomaly score or scores can be manually set and/or determined by the machine learning component 106 using the techniques described above.

The type machine learning models used for the anomaly detection models 114^{1-N} can vary. For example, the respective anomaly detection models 114^{1-N} can employ various types of machine algorithms, including (but not limited to): deep learning models, neural network models, deep neural network models (DNNs), convolutional neural network models (CNNs), generative adversarial neural network models (GANs), long short-term memory models (LSTMs), attention-based models, transformers, or a combination thereof. In some embodiments, the respective anomaly detection models 114^{1-N} can additionally or alternatively employ a statistical-based model, a structural based model, a template matching model, a fuzzy model or a hybrid, a nearest neighbor model, a naïve Bayes model, a decision tree model, a linear regression model, a k-means clustering model, an association rules model, a q-learning model, a temporal difference model, or a combination thereof. The machine learning component 106 can employ supervised, semi-supervised and/or unsupervised training methods for training the anomaly detection models 114^{1-N} based on the historical clinical data messages 124.

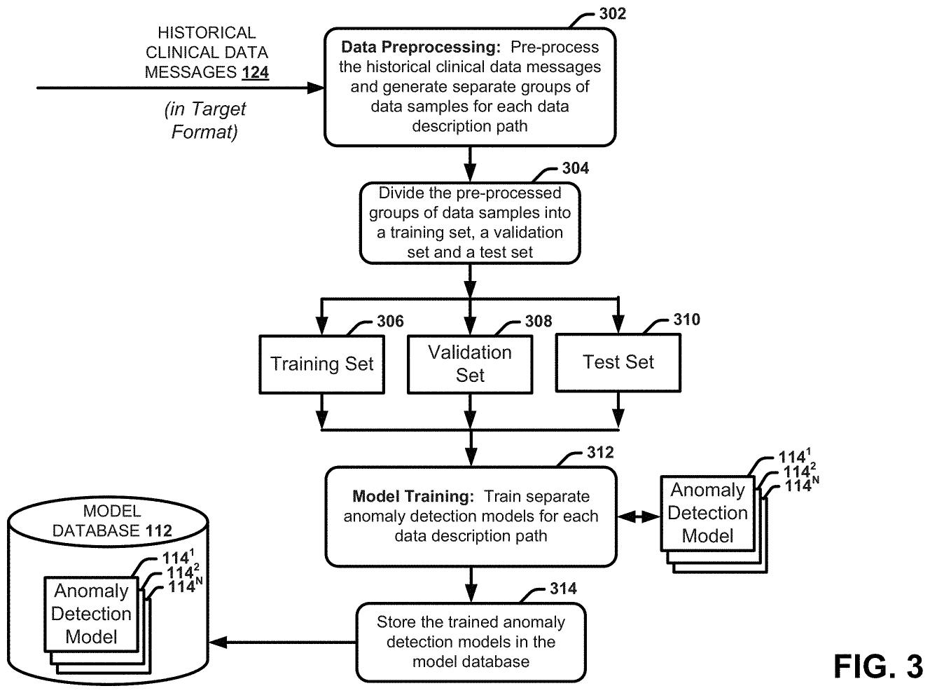

FIG. 3 illustrates a flow diagram of an example training process 300 for generating the anomaly detection models 114^{1-N} in accordance with one or more embodiments of the disclosed subject matter. In this regard, process 300 presents a high-level overview of an example training process that can be performed by the pre-processing component 104 and the training component 106 to train separate anomaly detection models 114 for each of the defined data description paths (e.g., each FHIR path/key) to generate anomaly scores for each of the defined data description paths that reflect a measure of the amount and/or severity of mapping errors associated with each data description path for a collection of new clinical data messages 126.

With reference to FIGS. 1 and 3, in accordance with process 300, at 302 the pre-processing component 102 can perform data pre-processing to prepare the historical clinical data messages 124 for model training and development. As described above, in some embodiments, this can involve segmenting the clinical data messages into separate groups of data samples for each data description path (e.g., each FHIR path/key). The data samples included in each group can comprise a collection of data samples belonging to each data description path of the defined set of data description paths. The data samples respectively correspond to data strings consisting of defined data elements in defined data fields, wherein one or more of the data fields corresponds to a value field. In some implementations, the pre-preprocessing at 202 can further involve extracting, for each data sample, the set of values (or single value in implementation in which a data description path includes one value data field) for each of the value data fields.

At 304, the pre-processing component 104 can further divide the pre-processed data samples into training set 306, a validation set 308 and a test set 310 in accordance with conventional machine learning training regimens using a training phase, a validation phase and a testing phase. In this regard, in accordance with conventional ML training techniques, the training set 308 is used during a model training phase to fit the respective anomaly detection models 114^{1-N}. The validation set 308 is used to provide an unbiased evaluation of the respective models fit on the training set 308 while tuning the models' parameters using a loss function. The evaluation becomes more biased as skill on the validation set 302 is incorporated into the models' configuration. In this regard, the validation loss is used to select the best version of the respective models generated during the training phase and avoid overfitting. The test set 310 is used to measure the performance of the versions of respective anomaly detection models 114^{1-N} selected during the validation phase.

At 312, the machine learning component 106 can perform model training, including the training phase, the validation phase and the testing phase. In this regard, at 312 the machine learning component 106 can train separate anomaly detection models 114^{1-N} for each data description path. In this regard, for each anomaly detection model 114^{1-N}, the training component 106 can train the corresponding models using their corresponding pre-processed group of data samples and/or extracted sets of values for the value fields. In various embodiments, at a high level, the training process at 312 can comprise training the respective anomaly detection models 114^{1-N} to estimate the likelihood that a new set of values mapped data for each data description path is normal, that is, the new set of values falls within an acceptable range of deviation from the training set. For example, in some embodiments, the machine learning component 106 can configure the anomaly detection models 114^{1-N} to generate anomaly scores for each of the data description paths given a new set of data samples and values. In some implementations of these embodiments, the machine learning component 106 can validate the respective anomaly detection models using new sets of data samples included in the validation set 308 taken from other data description paths. In various embodiments, the anomaly scores can reflect a measure of the distribution differences between the new values and the values of the corresponding set of data samples included in the training set 306. Once training is complete, at 314, the training component 306 can store the trained anomaly detection models 114^{1-N} in the model database 112.

In some embodiments, the machine learning component 106 can employ a deep learning tool known as a variational auto encoding (VAE) to learn an embedding of all string values into a common vector space. The idea is to learn an embedding space where all strings that “look the same” are mapped to a close subspace while such strings that are very different in shape are mapped far apart. With these embodiments, each of the anomaly detection models 114^{1-N} can comprise a VAE and the machine learning component 106 can train the respective VAEs using the historical clinical data messages 124 to learn the embedding space for each data description path. In particular, the machine learning 106 can train a separate VAE for each data description path (e.g., each FHIR path/key) based on the collection of training data samples for each data description paths and the values of the one or more value data fields included in each of the data description paths provided by the training data samples. During evaluation, given a new set of values per data description path, the anomaly detection component 108 can measure the average loss with respect to the corresponding model. The anomaly detection component 108 can further use the mean loss for each model to characterize the likelihood of the data sample conforming with the model.

To facilitate this end, in some embodiments, at 304 the pre-processing component 104 can index the distribution of values for each data field corresponding to a value for each data sample (e.g., each data string) belonging to each data description path. In this regard, assume M different data description paths (or FHIR paths/keys). The pre-processing component 104 can segment the messages into their respective data description paths (e.g., FHIR paths/keys) as described above, resulting in data samples corresponding to data strings for each data description path M. Said differently, the pre-processing component 104 can compute the JSON path keys per string value M. The pre-processing component 104 can further extract the string values corresponding to data field comprising values for each data description path M. The pre-processing component 104 can further map each of the data sample values or string value to an input vectors space of size N, wherein can vary. Said differently, the pre-processing component can split the string values over N. In some implements, N is arbitrarily selected to be 100. The pre-processing component 100 can further replace each character with its ASCII value which is normalized to the [0-1] domain. In some implementations, the pre-processing component 104 can normalize the ASCII values by a max ASCII value) and zero align them to N-vectors. The pre-processing component can further generate an M by N input table. For example, the pre-processing component 104 can create an indexed input table with N columns (e.g., 100) for zero padded data and a keys column (or data path description column) which associates each of the data paths to a different row (e.g., a separate row for each of the different data description path or FHIR path/key). In this regard, the pre-processing component can attach a class label per row corresponding to each data description path string.

During model training at 312, the machine learning component 106 can train the respective VAEs using the training set of the anomaly detection models 114^{1-N} using the training set so that set of values (e.g., or a single value in implementations in which a path only includes one value field) from each data description path (e.g., each FHIR path/key) are mapped via a distribution mapping function of each VAE near the mean of a multivariate gaussian distribution with small variance. The VAE for each data description path learns a deterministic transform G which maps these values to an embedding space and the parameters for mean and variance of a multivariate gaussian distribution in that space. During the validation phase or the testing phase, for each data description path, the machine learning component 106 passes the value set of a new data sample for through G, which is them mapped to the embedding space, and the corresponding anomaly detection model 114 computes its “likelihood” with respect to the trained multivariate gaussian distribution in that space (e.g., using a distance metric or another difference valuation metric). With these embodiments, data samples with a high likelihood (relative to a defined threshold) are considered as normal values while values with a low likelihood are considered as anomalous. In this regard, the trained multivariate gaussian distribution for a specific data description path (e.g., FHIR path/key) corresponds to a parameterized probability distribution of the acceptable value or set of values for that data description path and the “likelihood” represents the probability that a new value or set of values received for that data description path is included in or otherwise drawn from that parameterized probability distribution.

As a simple example, suppose we have simple gaussian distribution function for a small set of parameters, such as a mean 0 and variance 1 (the parameters). In accordance with this example, a new value of 0 will have an extremely high likelihood that it is drawn from that gaussian while a new value of 1000 will have an extremely low likelihood that it is not. In accordance with embodiments in which a data description path (e.g., an FHIR path/key) comprises a set (e.g., two or more) of values, the gaussian distribution function is multivariate (and thus much more complex) and parameterized by the weights of the neural network filters that form the VAE. These parameters form a mapping function which maps the item into an n-dimensional (multivariate) gaussian. In this regard, considering a new set of values received for a single data sample for a specific data description path, these values are mapped through its corresponding VAE and a measure of how close (e.g., in distance or another similarity metric) these values are to the normal multivariate distribution is computed. When the distance is large (e.g., beyond a threshold distance), this indicates the set of items was not taken from the same distribution that was used to train the VAE in the first place.

In various embodiments, the machine learning component 106 can train the respective VAEs using the variance of evidence lower bound (ELBO) loss function so that iteratively, the VAE's distribution mapping function forces mapped values to reside near the center of a multivariable gaussian, and that successively the parameters of the gaussian become sharper. In some embodiments, the machine learning component 106 can stop the training process when the trend in ELBO loss flattens (indicating convergence) and when the loss for a validation or test set starts to rise, signaling an overfitting. In this regard, during the validation or testing phase, for each data description path, a set of test data samples are computed with its corresponding VAE and the resulting ELBO loss values are averaged for all of the test data samples in the test set and considered the anomaly score of that set of data. For each VAE model which corresponds to a specific data description path (e.g., FHIR path/key), the average ELBO score is computed and the minimal value declared to be the class estimate for the set.

Additionally, or alternatively, instead of training one VAE per data description path, the machine learning component 106 can train one or more VAEs to learn a conditional relationship between the normal values for one or more pairs or groups (including three of more) of the defined data description paths. This is a result of an observation that in some cases an item might be identified as anomalous only within a context that is presented on a second data path. Specifically, in the case of FHIR, data pairs of items each taken from two different data path in the same FHIR bundle might be concatenated to form a single item that can be modeled in a pairwise VAE. For example, assume two paths A and B are related, wherein the value of the data element in field 6 of path A can vary and be valid or invalid depending on the value of the data element in field 7 of path B. For instance, consider the case of systolic or diastolic blood pressure (BP) wherein the value and type of these different data fields arrive in distinct data paths. To potentially detect the case where the value of systolic BP was mixed with diastolic BP, the machine learning component 107 can train a pairwise VAE that considers both the value and the type together and train an anomaly detection model (e.g., of the anomaly detection models 114^{1-N}) that employs the pairwise VAE to detect this pair. In this regard, for pairs of different data description paths with related data fields, the machine learning component 106 can train a pairwise VAE for both paths that accounts for the conditional probability that values mapped to the one path are valid based on the values mapped to the other path in the pair. For example, in the case of FHIR, data pairs of items each taken from two different data path in the same FHIR bundle might be concatenated to form a single item that the machine learning component 106 can model in a pairwise VAE. These pairs of items are taken from the same FHIR bundle. During training, the machine learning component 106 feeds the VAE these pairs and weighs the result according to the probability that the VAE produced the first item of the pair given the second item of the pair. During the evaluation phase, the machine learning component 106 can receive a new FHIR bundle, extract the corresponding two values for the first data path and the second data path and feed them into the pairwise VAE and compute the loss value (e.g., the ELBO loss value), wherein a large ELBO loss would indicate bad reconstruction and hence hint on anomalous pair of values. The machine learning component 106 can generate any number of VAE in this manner designed to account for the conditional relationship between values in two or more related or dependent data description paths.

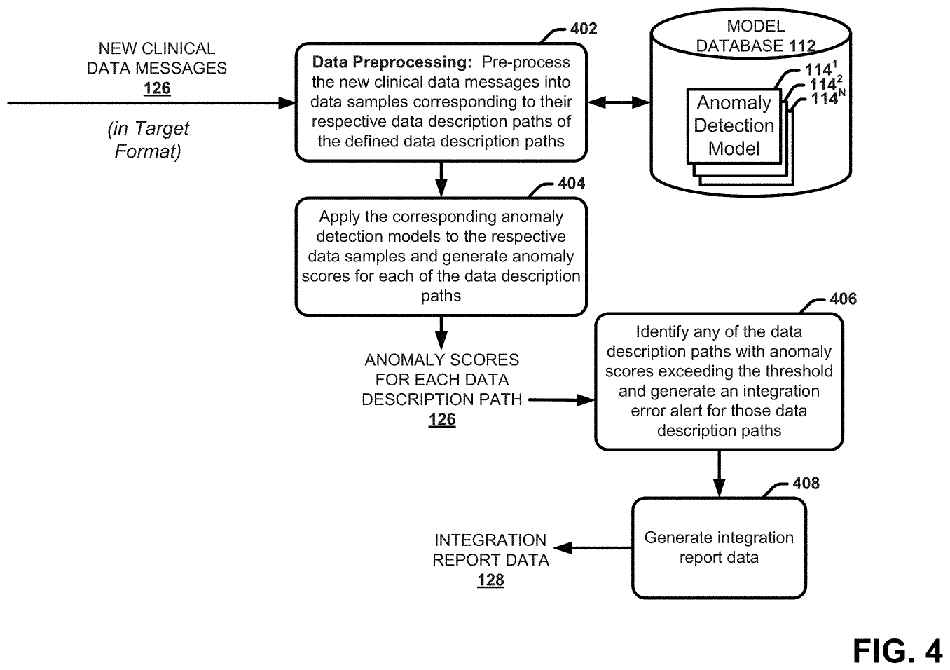

Once the machine learning component 106 can completed training the anomaly detection models 114^{1-N}, the anomaly detection component 108 can apply the anomaly detection models to the new clinical data messages 126 to determine anomaly scores for each of the defined data description paths, as illustrated in FIG. 4 and process 400

In this regard, FIG. 4 illustrates a flow diagram of an example process 400 that can be performed by the clinical data integration system 100 using the pre-processing component 104, the anomaly detection component 108, the alert component 116 and the reporting component 118 to detect and report data discrepancies associated with clinical data integration in accordance with one or more embodiments of the disclosed subject matter.

With reference to FIGS. 1 and 4, in accordance with process 400, after reception of the new clinical data messages by the reception component 102, at 402, the new clinical data messages 126 can be pre-processed by the pre-processing component 104 using the same techniques used to pre-preprocess the historical clinical data messages 124. For example, the pre-processing component 104 can pre-process the new clinical data messages 126 into data samples corresponding to their respective data description paths of the defined data description path using the techniques described above. For example, in implementations in which the new clinical data messages 126 are mapped from their one or more native formats into the FHIR format in JSON and the defined data description paths correspond to FHIR paths or keys, each new clinical data messages 126 can be broken down into its respective FHIR paths or keys, wherein the respective FHIR paths or keys correspond to the path from the root of each JSON object included in each of the new clinical data messages. The pre-processing component 104 can further group data samples belonging to the same data description path (e.g., FHIR path/key) together. In some implementations, for each data sample, the pre-processing component 124 can further identify and extract the values for each of the value data fields included in each of the data description paths. The pre-processing component 104 can further perform any of the additional pre-processing functions described with reference to the historical data messages 124 to the new clinical data messages 126.

At 404, the anomaly detection component 108 can apply the corresponding anomaly detection models 114^{1-N} to the respective data samples and generate anomaly scores for each for the data description paths 126. For example, in implementations in which the anomaly detection models 114^{1-N} comprise VAEs trained for each of the data description paths, the anomaly detection component 108 can pass each data sample through its corresponding VAE to determine an anomaly score for each data sample as a function of the loss value (e.g., the ELBO loss) with respect to the corresponding VAE trained multivariate gaussian space. The anomaly detection component 108 can further determine the anomaly scores for each of the represented data description path (e.g., each FHIR path/key) based on the average anomaly score (or loss values) for all of the individual data samples belonging to each represented data description path. In this regard, if the new clinical data messages 126 correspond to messages for a new integration project coming from a new set of clinical information systems for a new hospital site, some of the defined data description paths may not be included in the new clinical data messages.

At 406, the alert component 116 can identify any of the data description paths with anomaly scores exceeding the threshold anomaly score (a general threshold or a separate threshold tailored for each data description path) and generate an integration error alert for those data description paths. At 408, the reporting component 118 can generate the integration report data 128 based on the results of the anomaly detection models 114^{1-N}. The reporting component 118 can further provide the integration report data to integration engineers to facilitate reviewing detected mapping errors. For example, the reporting component 118 can generate the integration report data 128 in human interpretable format that can be rendered via a suitable display device. In some embodiments, the integration report data 128 can include a list of all the defined data description paths and anomaly scores determined for each of the defined data description paths that indicate a measure of the amount and/or severity of the detected mapping errors associated with each of the data description paths. The integration report data 128 can also include one or more representative data samples and/or links thereto for each of the defined data description paths associated with anomaly scores that exceed maximum threshold anomaly score and thus are considered to be likely to include an unacceptable level of mapping errors. The representative data samples can include all of the associated data samples for that path whose individual anomaly scores exceed the threshold, or a select subset. For example, the select subset can include the top T data samples with the highest anomaly scores (e.g., top 10), a range of data samples with varying anomaly scores over the threshold, or another select subset. In some implementations, the representative data samples may also include one or more correctly mapped data samples (whose anomaly scores were below the threshold) for any of the defined description paths.

As noted above, in some embodiments, process 400 can performed by the clinical data integration evaluation system 100 to detect mapping errors associated with new clinical data messages 126 which correspond to mapped data messages for a new integration project involving a new set of clinical information systems for a new hospital (or a similar system) using the same previously defined mapping function (or a different mapping function). In this regard, the new integration project may include one or more different clinical information systems that provide the new clinical data messages. In addition, the content of the contents of the new clinical data messages 126 may vary to reflect the clinical data associated with the new hospital and the one or more native formats in which the clinical information systems generate, store and/or report the clinical data may vary. The types of the clinical data message and their contents may also vary for the new clinical data integration project based on a particular consuming application for which the new integration project is based.

In one example usage illustration, arriving to a new site and completing an initial data “plumbing” phase, all data starts to flow from the new clinical information sources in their native formats and are mapped into the target format (e.g., FHIR or another target format) via the same mapping function used to generate the training data (i.e., the historical clinical data messages 124). Assuming the target format is FHIR, a corpus of mapped FHIR messages is collected and such that it represents normal data production of the new hospital by the new clinical information sources (e.g., for multiple patients over the duration of several days). In accordance with this example usage illustration, the corpus of mapped FHIR messages can correspond to the new clinical data messages 126. Alternatively, any existing data dumps may also be used to collect the new clinical data messages 126. With these embodiments, the previously defined native to target format mapping function of the previously successful integration project from which the training data was generated can be used as a starting point in subsequent integrations projects, and process 400 can be utilized to identify any of data description paths associated with mapping errors. An operating technician further adapted the mapping rules for the new integration project based on the identified data description paths with mapping errors, focusing only on those mapping rules associated with the data description paths and associated value data fields that need adapting while leaving those mapping rules associated with valid data description paths in place. As a result, the process for generating new mapping rules for a new integration project can be significantly reduced.

Additionally, or alternatively, the clinical data integration evaluation system 100 can employ the trained anomaly detection models 114^{1-N} to continuously monitor an ongoing stream of messages and detect and report mapping errors in real-time or substantially real-time. With these embodiments, the new clinical data messages 126 can correspond to those associated with a new integration project (e.g., for a new hospital system, a new set of clinical information systems, and/or a new clinical application) or from the same system/site from which the training data was generated (e.g., the historical clinical data messages 124) to detect mapping errors that may arise over time due to configuration changes, system updates, and other factors. With these embodiments, the new clinical data messages 126 can be received and processed by clinical data integration system 100 in real-time to generate per message anomaly scores in real-time. For example, assume the new clinical data messages 128 correspond to a live stream of clinical data messages transmitted from one or more clinical information systems to clinical application for processing thereof. In this context, the new clinical data messages 126 are intercepted by the mapping/conversion engine that applies the predefined mapping function to convert them into the target format. In addition, the clinical data integration evaluation system 100 can process each converted message as it is received to detect mapping errors in real-time.

In this regard, for each received message, the clinical data integration evaluation system can break the message into data samples (e.g., data strings) corresponding to the respective data description paths of the defined data description path that are included in the message. The system can further generate anomaly scores for each of the included data description paths based on the corresponding data samples using one or more of the techniques disclosed herein. In some implementations, the alert component 116 can further identify any of data description paths associated with that message whose anomaly scores exceed the defined threshold anomaly score and generate an integration error alert for those data description paths in real-time. Additionally, or alternatively, the respective data description path anomaly scores can further be continuously and/or regularly averaged and updated in real-time based on the received messages. With these embodiments, the alert component 116 can be configured to generate integration error alerts based on the average anomaly scores associated with the same path exceeding the threshold and based on some restrictions regarding the number of data samples received for a given path and/or the frequency of received data samples. Alternatively, the clinical data integration evaluation system 100 can be configured to aggregate and store collections of the new incoming clinical data messages and apply the anomaly detection models to new sets of the aggregated messages according to a defined scheduled (e.g., every hour, every 24 hours, every 48 hours, every week, etc.) and the alert component 116 can generate integration error alerts based on the average anomaly scores determined for each data description path aggregated each defined time frame.

With these embodiments, the reporting component 118 can further report the integration error alerts in real-time (e.g., in response to detection and generation thereof). For example, the reporting component 118 can generate real-time notifications regarding any detected integration error alerts that can be presented via a graphical user interface employed by the clinical data integration evaluation system 100 to provide the integration report data 128 to end users (e.g., operating technicians or the like). One example of such graphical user interface is provided in FIGS. 7A-7B. Additionally, or alternatively, the reporting component 118 can generate and provide such real-time notifications regarding any detected integration error alerts to the clinical application (e.g., as an application notification or the like).

As described herein, a real-time computer system can be defined a computer system that performs its functions and responds to external, asynchronous events within a defined, predictable (or deterministic) amount of time. A real-time computer system such as system 100 and other system described herein (e.g., system 500 and/or system 600) typically controls a process (e.g., detecting integration mapping errors) by recognizing and responding to discrete events within predictable time intervals, and by processing and storing large amounts of data acquired from the controlled system (e.g., the new clinical data messages 126). Response time and data throughput requirements can depend on the specific real-time application, data acquisition and critical nature of the type of decision support provided. In the regard, the term “real-time” as used herein with reference to processing the new clinical data messages 126 to detect integration errors and generating corresponding alerts refers to performance of these actions within a defined or predictable amount of time (e.g., a few seconds, less than 10 seconds, less than a minute, etc.) between reception of the new clinical data messages 126. Likewise, the term real-time as used with reference to reception of the new clinical data messages 126 refers to reception of the new clinical data messages 126 from the mapping/conversion engine within a defined or predictable amount of time (e.g., a few seconds, less than 10 seconds, less than a minute, etc.) after the corresponding information transmitted to or otherwise received by the mapping/conversion engine from the respective clinical information systems.

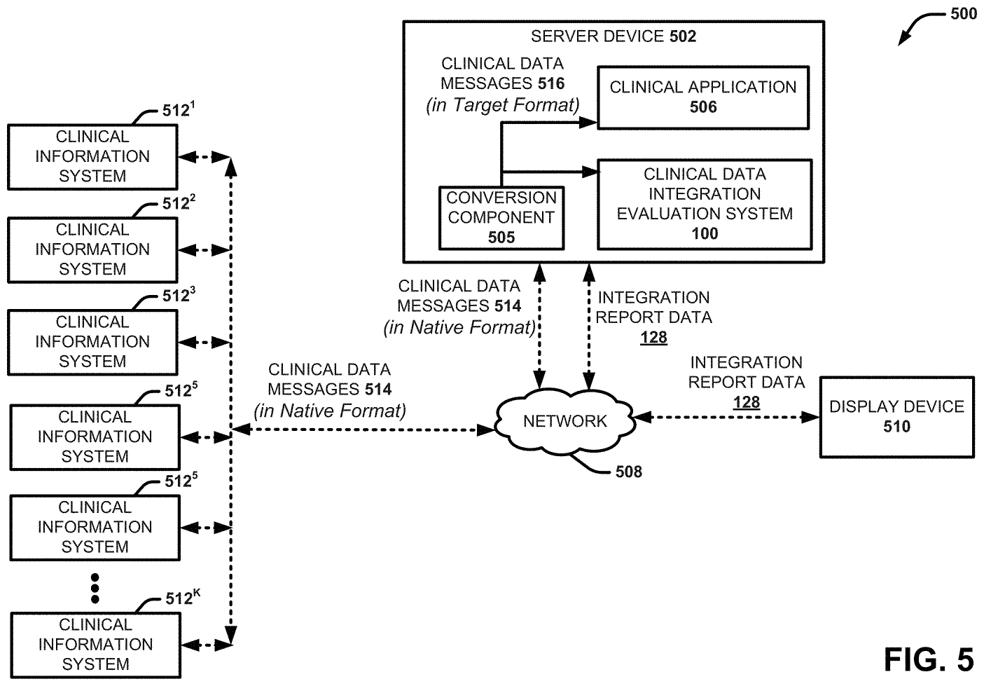

FIG. 5 presents another example, non-limiting system 500 that facilitates detecting data discrepancies associated with clinical data integration in accordance with one or more embodiments of the disclosed subject matter. System 500 provides an example system architecture in which the clinical data integration evaluation system 100 may be implemented. Repetitive description of like elements employed in respective embodiments is omitted for sake of brevity.

In various embodiments, the clinical data integration evaluation system 100 can be integrated into the medical data ingestion pipeline between the integration engine the performs the data format conversion mapping and the entity that consumes the converted clinical data in the target format, such as a clinical application. For example, in accordance with system 500, the clinical data integration evaluation system 100 can be deployed at a centralized sever device 502 that is communicatively coupled to a plurality of different clinical information systems 512^{1-K} via a network 508. The server device 502 can also include the clinical application 506 that corresponds to an application configured to consume the clinical data messages 516 in the target format. Additionally, or alternatively, the clinical application 506 can be deployed at a separate system or device (other than the server device 502). The network 508 can be a communication network, a wireless network, an internet protocol (IP) network, a voice over IP network, an internet telephony network, a mobile telecommunications network and/or another type of network. The server device 502 can also include a conversion component 504 that can correspond to an integration engine that employs defined mapping logic (e.g., mapping rules and translation tables) to convert clinical data messages 514 generated in one or more native formats into the target data format for processing by the clinical data integration evaluation system 100 and in some implementations, the clinical application 506. Additionally, or alternatively, the conversion component 404 can be included in the clinical data integration evaluation system 100 or deployed at separate system or device (other than the server device 502).

In this regard, in some implementations the clinical data messages 516 in the target format can correspond to the historical clinical data messages 124 or the new clinical data messages 126. In some implementations in which the clinical data messages 516 correspond to the new clinical data messages 126, the set of clinical information systems 512^{1-K} may be different and/or associated with a different hospital system from which the training data (e.g., the historical clinical data messages 124) was received, and thus produce different messages than the system used to generate the historical clinical data messages 124. In some implementations in which the clinical data messages 516 correspond to the new clinical data messages 126, the mapping function used by the conversion component 504 can correspond to the same mapping function used to generate the historical clinical data messages 124 in association with a previously successful integration project and tailored to a different clinical application and/or set of clinical information systems providing the new clinical data messages.

System 500 further includes a display device 510 that is also communicatively coupled to the sever device 502 via the network 508. The display device 510 can correspond to any suitable computing device capable of receiving and rendering integration report data 128 generated by the clinical data integration evaluation system 100. The display device 510 can further include suitable hardware and software for accessing the clinical application 506 and the clinical data integration evaluation system 100 via the network 508, rendering an interactive graphical user interface that includes the integration report data 128 and enabling user interaction with the graphical user interface (e.g., via one or more suitable input devices/mechanism). For example, the display device 510 can correspond to a device used by an operating technician responsible for evaluating the integration report data 128 and employing the integration report data 128 to facilitate adjusting the mapping function used by the conversion component 504 to remediate detected mapping errors. Additionally, or alternatively, the clinical event data monitoring system 100 can provide the integration report data 128 via the clinical application 506 on another system/device. In this regard, the display device 510 can be a mobile device, a mobile application for a mobile device, a wall display, a monitor, a computer, a tablet computer, a wearable device, and/or another type of display device.

The clinical information systems 512^{1-K} can correspond to a variety of different electronic information systems, devices, databases, data sources and the like configured to generate, store, report, transmit and/or otherwise provide the clinical data messages 514 for usage by the clinical application 506 (or another clinical application). The number of different clinical information systems 512^{1-K} can vary, the type and contents of the clinical event data messages 514 can vary, and the native format or formats used by the clinical information systems to generate the clinical data messages 514 can vary. For example, one or more of the clinical information systems 512^{1-K} may include, but are not limited to, one or more patient electronic health record (EHR) systems, one or more patient monitoring systems/devices, one or more bed management systems, one or more medical imaging systems, one or more laboratory systems, one or more facility operations tracking system, one or more medication management systems, one or more admission/discharge recording system, one or more clinical ordering systems, one or more clinical billing systems, and various other electronic medical facility information sources/systems.

It should be appreciated that the various types of clinical information systems describe above are merely exemplary and other or alternative types of healthcare related data sources/system are envisioned that may provide the clinical data messages 514.

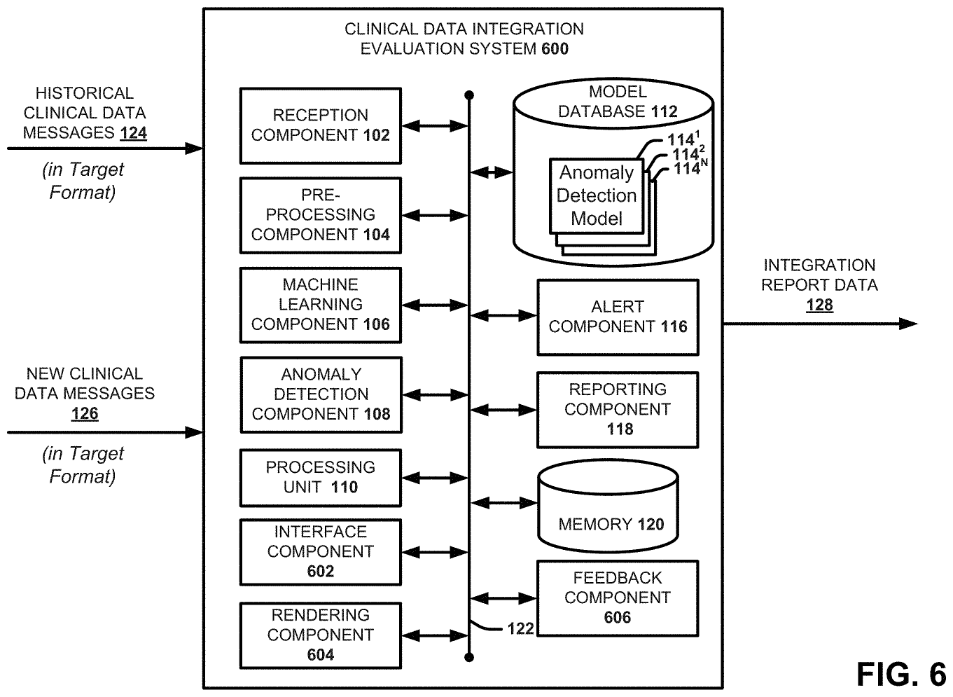

FIG. 6 illustrates another example, non-limiting clinical data integration evaluation system 500 that facilitates detecting data discrepancies associated with clinical data integration in accordance with one or more embodiments of the disclosed subject matter. Clinical information evaluation system 600 can include same or similar components as clinical information evaluation system 100 with the addition of interface component 602, rendering component 604 and feedback component 606. Repetitive description of like elements employed in respective embodiments is omitted for sake of brevity.

In one or more embodiments, the interface component 602 and the rendering component 604 can facilitate providing the integration report data 128 to integration engineers for their review in human interpretable format that can be rendered via a suitable display device (e.g., display device 510). For example, in some implementations, the rendering component 604 can be operatively and/or communicatively coupled to the display device and render the integration report data via a graphical display. The integration report data 128 can include a list of all the defined data description paths (e.g., FHIR paths/keys) and anomaly scores determined for each of the defined data description paths that indicate a measure of the amount and/or severity of the detected mapping errors associated with each of the data description paths. The integration report data 128 can also include one or more representative data samples for defined data description paths. In some implementations, the alert component 116 can flag (e.g., with an alert notification icon or symbol) any of the data description paths associated with an anomaly score that exceeds a maximum anomaly score threshold (e.g., a universal threshold applied to all paths or tailored thresholds for each path) and thus are considered to be likely to include an unacceptable level of mapping errors. In some implementations, the anomaly score threshold or thresholds associated with each data description path can also be included in the integration report data.

In some embodiments, interface component 602 can generate an interactive graphical user interface comprising the integration report data 128 and provide corresponding interaction logic that provides for interacting with the integration report data via the graphical user interface to facilitate evaluating the integration report data 128 to gain a better understanding of the potential mapping errors and performing root cause analysis to determine potential causes of the mapping errors. For example, the interactive graphical user interface can include a scrollable list of all the defined data description paths and their anomaly scores, which may be selectable, wherein upon selection, the interface component 602 can present additional information regarding the results of the anomaly detection model associated with each path and include selectable representative data samples associated with each path. The interactive graphical user interface can further provide a mechanism for searching and filtering the data description paths based on various parameters (e.g., anomaly scores, data description path types or class labels, data field types, etc.), and adjusting the anomaly score threshold or thresholds. The interactive graphical user interface and the feedback component 606 can further provide a mechanism for receiving user feedback regarding the accuracy of the anomaly scores as determined based on manual review of the representative data samples. For example, after investigating representative data samples for a data description path that received a significantly high anomaly score, the integration engineer may decide that the data samples were actually mapped correctly and thus provide feedback indicating the anomaly score for that data description path is not accurate. In this regard, the feedback component 606 can receive user feedback for any of the defined data description paths (e.g., FHIR paths/keys) that indicates a measure of the accuracy of the anomaly score associated therewith and/or the anomaly scores of individual data samples. The feedback component 606 can further aggregate any received feedback for each data description path regarding the accuracy of the anomaly detection model associated therewith. The machine learning component 106 can further perform a continuous learning regime using the feedback to retrain and update (e.g., fine tune) the corresponding anomaly detection models over time.

FIGS. 7A-7B present an example graphical user interface that facilitates reviewing integration report and receiving user feedback regarding the accuracy of the anomaly detection models in accordance with one or more embodiments of the disclosed subject matter. The graphical user interface corresponds to an example graphical user interface that can be generated by the interface component 602 based on the integration report data 128 and presented to an operating technician via the rendering component 604 via a display device including input capabilities. In this example, the graphical user interface provides the results of the anomaly detection component 108 (i.e., the integration report data 128) after application of the anomaly detection models 114^{1-N} to a new set of clinical data messages (e.g., new clinical data messages 126) following completion of model training. The interactive graphical user interface provides interactive tools for reviewing the results and receiving user feedback regarding the accuracy of the results. The combined functionalities of the interactive graphical user interface and the anomaly detection component may be integrated into a suitable user application/tool, referred to in this example as the “Conversion Analyzer.”

As illustrated in FIGS. 7A-7B, the interactive graphical user interface can include scrollable list 702 of each of the defined data description paths for which an anomaly detection model was trained. In this example, the data description paths correspond to FHIR resource paths (also referred to as FHIR keys or keys for short). The interface further provides the mapping error scores 704 determined for each of the FHIR resource paths. These mapping error scores can correspond to the respective anomaly scores determined for each FHIR resource path based on the average anomaly scores generated for each associated data sample via the corresponding anomaly detection models. In this example, those scores associated with a value greater than a 1.0 score threshold value are flagged (e.g., by the alert component 116) as including or potentially including mapping errors. The conversion analyzer further provides an upper toolbar 706 with tools for manually adjusting this score threshold, referred to in this example as the credibility threshold. After adjusting, the “estimate mapping error” icon can be selected to apply the adjusted threshold and change the corresponding fagged FHIR resource paths. The upper toolbar 706 also include options to filter the FHIR resource path based on missing keys, which correspond to those FHIR resource paths for which no new data samples were received, and processed keys, which correspond to those FHIR resource paths for which new data samples were received. As illustrated in FIG. 6B, the conversion analyzer also includes information regarding the mapping distance 708 associated with each FHIR resource path and the internal disparity level 710. The conversion analyzer further includes a “view sample” option that upon selection, presents a list of one or more representative samples for the corresponding paths. The conversion analyzer further include a feedback selection function 714 via which the user can provide feedback regarding whether they agree the mapping associated with each path is correct or incorrect, which can be determined by the reviewer based on review of the representative data samples. In this regard, “correct mapping” icon can be selected to indicate the mapping associated with a corresponding path is correct and the “failed mapping” icon can be selected to indicate the mapping is incorrect. After selecting these feedback icons, the “save classifications” icon at in the upper toolbar 706 can be selected to save the feedback received for each path. This feedback can be aggregated by the feedback component 606 for each path and used by the machine learning component 106 to update the corresponding anomaly detection models over time (e.g., via retraining and fine tuning the models).

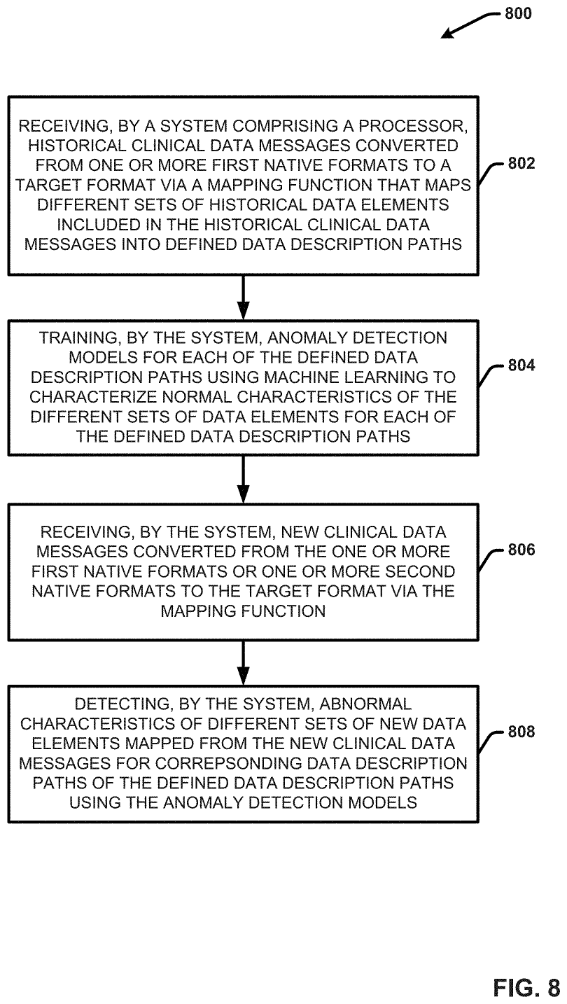

FIG. 8 illustrates a high-level flow diagram of an example method 800 for detecting data discrepancies associated with clinical data integration in accordance with one or more embodiments of the disclosed subject matter. Repetitive description of like elements employed in respective embodiments is omitted for sake of brevity.

In accordance with method 800, at 802, a system operatively coupled to a processer (e.g., system 100, system 500, system 600, or the like) receives historical clinical data messages converted from one or more first native formats to a target format via a mapping function that maps different sets of historical data elements included in the historical clinical data messages into defined data description paths (e.g., via reception component 102). At 804, the system trains anomaly detection models (e.g., anomaly detection models 114^{1-N}) for each of the defined data description paths using machine learning to characterize normal characteristics of the different sets of data elements for each of the defined data description paths. At 806, the system receives new clinical data messages converted from the one or more first native formats or one or more second native formats to the target format via the mapping function (e.g., via reception component 102). At 808, the system detects abnormal characteristics of different sets of new data elements mapped from the new clinical data messages for corresponding data description paths of the defined data description paths using the anomaly detection models (e.g., via the anomaly detection component 108). In this regard, it should be noted that the new clinical data messages may not include data samples belonging to each of the defined data description paths represented in the historical clinical data messages (i.e., the training data). Thus, at runtime, the system will apply only those data detection models for the data description paths that the new clinical data messages are mapped to.

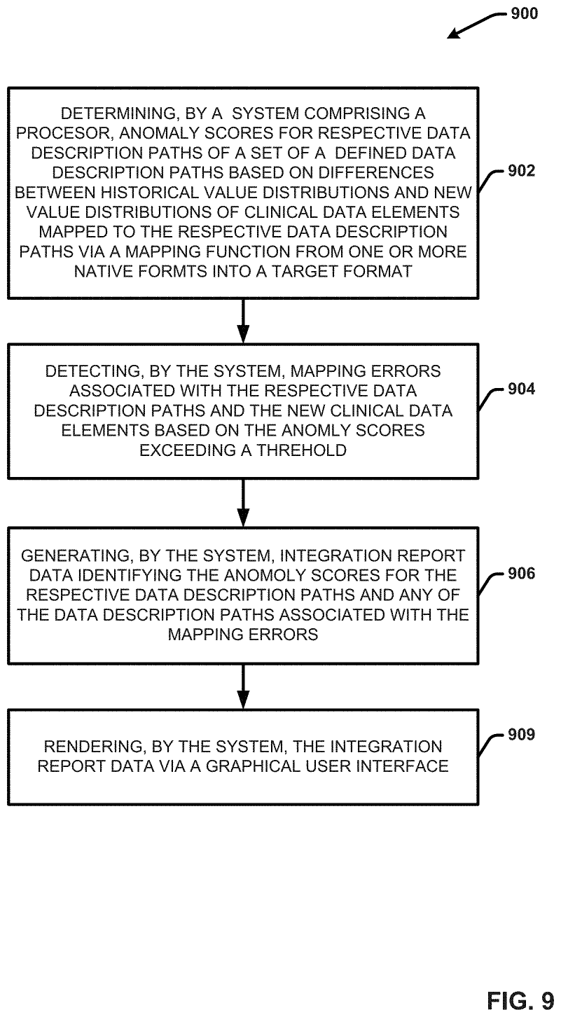

FIG. 9 illustrates a high-level flow diagram of another example method 900 for detecting data discrepancies associated with clinical data integration in accordance with one or more embodiments of the disclosed subject matter. Repetitive description of like elements employed in respective embodiments is omitted for sake of brevity.

In accordance with method 900, at 902, a system operatively coupled to a processer (e.g., system 100, system 500, system 600, or the like) determines anomaly sores for respective data description paths of a set of defined data description paths based on differences between distributions between historical value distributions and new value distributions of clinical data elements mapped to the respective data description paths via a mapping function from one or more native formats into a target format (e.g., via the detection component 108 using the corresponding anomaly detection models 114^{1-N} for each of the defined data description paths).

For example, in some embodiments, the machine learning component 106 can compute the historical value distributions for each path based on historical clinical data messages using histogram representations and store them as reference representations of the normal distribution of values for each path. At runtime, the anomaly detection component 106 can further compute new histogram representation of the value distributions for the new clinical data elements for the corresponding paths, compare the new histogram representation to the corresponding reference histogram representations, and determine the anomaly scores based on the differences (e.g., a distance difference determined using a distance metric such as the Bhattacharyya distance or another distance metric) between the reference and new histogram representations. In other embodiments, the machine learning component 106 can train anomaly detection models 114^{1-N} for each of the defined data description paths based on the historical clinical data messages to learn the normal distributions of values for each of the defined data description paths. The machine learning component 106 can further configure the anomaly detection models to process new data samples mapped to the corresponding data description paths via the same mapping function (or a different mapping function) and generate anomaly scores for the corresponding data description paths. For example, in some implementations, the machine learning component 106 can train VAEs for each of the defined data description paths to learn an embedding space of all string values mapped to the respective paths from the historical clinical data messages. With these embodiments, each of the anomaly detection models 114^{1-N} can include a separately trained VAE. At runtime, the anomaly detection component 106 can pass new data samples for the data description paths through their corresponding VAEs which map them to the learned embedding space. The machine learning component 106 can further configure the anomaly detection models 114^{1-N} to determine a measure (e.g., an ELBO loss value or another measure) of the likelihood that a new data sample conforms to the learned embedding space for its corresponding data description path and determine an anomaly score for that data sample based on this measure. computed with respect to the trained multivariate gaussian. The anomaly detection component 108 can further compute an anomaly score for each data description path based on the average anomaly scores computed for each received data sample belonging to that data description path. Additionally, or alternatively, one or more of the VAEs can account for pairs or groups of data description paths and model the conditional probability of values in one path as being valid based on the values in another path (or paths).

At 904, the system detects mapping errors associated with the respective data description paths based on the anomaly scores exceeding a threshold (e.g., via the detection component 108 and/or the alert component 116). At 906, the system generates integration report data identifying the anomaly scores for the respective data description paths associated with the mapping errors (e.g., via the reporting component 118). At 908, the system renders the integration report data via a graphical user interface (e.g., via interface component 602 and/or rendering component 604).

One or more embodiments of the disclosed techniques provide a data dependent approach to the process of clinical data integration. By using machine learning to train anomaly detection models 114^{1-N}, the disclosed techniques structure a mechanism for knowledge transfer from current successful integrations to subsequent integrations. The proposed approach further provides a semi-supervised continuous training regime wherein manual feedback from the operating engineers (i.e., or a similar subject matter expert) are used to continuously improve said models. By using the data from successful integrations to train models for subsequent ones, this approach circumvents one of the major obstacles in machine learning, that is the difficulty in obtaining labeled training data for model training.

The disclosed techniques further significantly reduce the complexity of clinical data integration for new integrations projects for new hospitals and/or new applications by creating an automated mapping error detection and alerting tool that significantly shortens the time for completing a data integration project by highlighting possible integration discrepancies to integration engineers. Reducing the effort of such projects is expected to lower the barrier of entry for such applications. In this regard, by highlighting specific data description paths where data discrepancies are suspected, the integration engineers are able to focus only on those suspected items. By accelerating the process of detecting data discrepancies, integration engineers can become more effective and ongoing integration project maintenance costs can be significantly reduced over time. In addition, less experienced integration engineers can become more effective which can reduce the overall cost of an integration project.

Many technical advantages for this approach are realized due to it being a data driven approach instead of a knowledge driven approach. In this regard, instead of manually encoding which values are valid and which are not for every converted data string, the proposed approach learns how normal data should look like and is able to give a probabilistic estimate to the level of normality a set of data items exhibit. By thresholding this normality score data, discrepancies can be automatically identified and flagged.

The disclosed techniques further use an anomaly detection framework of thinking which offers a more robust approach to handling unknown data discrepancies than a knowledge-based approach. Specifically, unseen values may be handled more uniformly than a knowledge-based alerting system would allow. For example, an alerting system based on regular expressions would only capture errors that it was designed for. In addition, the integration of a semi-supervised approach into the system allows it to continuously improve the anomaly detection models and offers a built-in method for improving system performance over time.

One or more embodiments can be a system, a method, and/or a computer program product at any possible technical detail level of integration. The computer program product can include a computer readable storage medium (or media) having computer readable program instructions thereon for causing a processor to carry out one or more aspects of the present embodiments.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the entity's computer, partly on the entity's computer, as a stand-alone software package, partly on the entity's computer and partly on a remote computer or entirely on the remote computer or server. In the latter scenario, the remote computer can be connected to the entity's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It can be understood that each block of the flowchart illustrations and/or block diagrams, and combinations of blocks in the flowchart illustrations and/or block diagrams, can be implemented by computer readable program instructions.

These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession may, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

In connection with FIG. 10, the systems and processes described below can be embodied within hardware, such as a single integrated circuit (IC) chip, multiple ICs, an application specific integrated circuit (ASIC), or the like. Further, the order in which some or all of the process blocks appear in each process should not be deemed limiting. Rather, it should be understood that some of the process blocks can be executed in a variety of orders, not all of which can be explicitly illustrated herein.

With reference to FIG. 10, an example environment 1000 for implementing various aspects of the claimed subject matter includes a computer 1002. The computer 1002 includes a processing unit 1004, a system memory 1006, a codec 1035, and a system bus 1008. The system bus 1008 couples system components including, but not limited to, the system memory 1006 to the processing unit 1004. The processing unit 1004 can be any of various available processors. Dual microprocessors and other multiprocessor architectures also can be employed as the processing unit 1004.

The system bus 1008 can be any of several types of bus structure(s) including the memory bus or memory controller, a peripheral bus or external bus, or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 13104), and Small Computer Systems Interface (SCSI).

The system memory 1006 includes volatile memory 1010 and non-volatile memory 1012, which can employ one or more of the disclosed memory architectures, in various embodiments. The basic input/output system (BIOS), containing the basic routines to transfer information between elements within the computer 1002, such as during start-up, is stored in non-volatile memory 1012. In addition, according to present innovations, codec 1035 can include at least one of an encoder or decoder, wherein the at least one of an encoder or decoder can consist of hardware, software, or a combination of hardware and software. Although, codec 1035 is depicted as a separate component, codec 1035 can be contained within non-volatile memory 1012. By way of illustration, and not limitation, non-volatile memory 1012 can include read only memory (ROM), programmable ROM (PROM), electrically programmable ROM (EPROM), electrically erasable programmable ROM (EEPROM), Flash memory, 3D Flash memory, or resistive memory such as resistive random access memory (RRAM). Non-volatile memory 1012 can employ one or more of the disclosed memory devices, in at least some embodiments. Moreover, non-volatile memory 1012 can be computer memory (e.g., physically integrated with computer 1002 or a mainboard thereof), or removable memory. Examples of suitable removable memory with which disclosed embodiments can be implemented can include a secure digital (SD) card, a compact Flash (CF) card, a universal serial bus (USB) memory stick, or the like. Volatile memory 1010 includes random access memory (RAM), which acts as external cache memory, and can also employ one or more disclosed memory devices in various embodiments. By way of illustration and not limitation, RAM is available in many forms such as static RAM (SRAM), dynamic RAM (DRAM), synchronous DRAM (SDRAM), double data rate SDRAM (DDR SDRAM), and enhanced SDRAM (ESDRAM) and so forth.

Computer 1002 can also include removable/non-removable, volatile/non-volatile computer storage medium. FIG. 10 illustrates, for example, disk storage 1014. Disk storage 1014 includes, but is not limited to, devices like a magnetic disk drive, solid state disk (SSD), flash memory card, or memory stick. In addition, disk storage 1014 can include storage medium separately or in combination with other storage medium including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage 1014 to the system bus 1008, a removable or non-removable interface is typically used, such as interface 1016. It is appreciated that disk storage 1014 can store information related to an entity. Such information might be stored at or provided to a server or to an application running on an entity device. In one embodiment, the entity can be notified (e.g., by way of output device(s) 1036) of the types of information that are stored to disk storage 1014 or transmitted to the server or application. The entity can be provided the opportunity to opt-in or opt-out of having such information collected or shared with the server or application (e.g., by way of input from input device(s) 1028).

It is to be appreciated that FIG. 10 describes software that acts as an intermediary between entities and the basic computer resources described in the suitable operating environment 1000. Such software includes an operating system 1018. Operating system 1018, which can be stored on disk storage 1014, acts to control and allocate resources of the computer system 1002. Applications 1020 take advantage of the management of resources by operating system 1018 through program modules 1024, and program data 1026, such as the boot/shutdown transaction table and the like, stored either in system memory 1006 or on disk storage 1014. It is to be appreciated that the claimed subject matter can be implemented with various operating systems or combinations of operating systems.

An entity enters commands or information into the computer 1002 through input device(s) 1028. Input devices 1028 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 1004 through the system bus 1008 via interface port(s) 1030. Interface port(s) 1030 include, for example, a serial port, a parallel port, a game port, and a universal serial bus (USB). Output device(s) 1036 use some of the same type of ports as input device(s) 1028. Thus, for example, a USB port can be used to provide input to computer 1002 and to output information from computer 1002 to an output device 1036. Output adapter 1034 is provided to illustrate that there are some output devices 1036 like monitors, speakers, and printers, among other output devices 1036, which require special adapters. The output adapters 1034 include, by way of illustration and not limitation, video and sound cards that provide a means of connection between the output device 1036 and the system bus 1008. It should be noted that other devices or systems of devices provide both input and output capabilities such as remote computer(s) 1038.

Computer 1002 can operate in a networked environment using logical connections to one or more remote computers, such as remote computer(s) 1038. The remote computer(s) 1038 can be a personal computer, a server, a router, a network PC, a workstation, a microprocessor based appliance, a peer device, a smart phone, a tablet, or other network node, and typically includes many of the elements described relative to computer 1002. For purposes of brevity, only a memory storage device 1040 is illustrated with remote computer(s) 1038. Remote computer(s) 1038 is logically connected to computer 1002 through a network interface 1042 and then connected via communication connection(s) 1044. Network interface 1042 encompasses wire or wireless communication networks such as local-area networks (LAN) and wide-area networks (WAN) and cellular networks. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 1044 refers to the hardware/software employed to connect the network interface 1042 to the bus 1008. While communication connection 1044 is shown for illustrative clarity inside computer 1002, it can also be external to computer 1002. The hardware/software necessary for connection to the network interface 1042 includes, for exemplary purposes only, internal and external technologies such as, modems including regular telephone grade modems, cable modems and DSL modems, ISDN adapters, and wired and wireless Ethernet cards, hubs, and routers.

The illustrated aspects of the disclosure may also be practiced in distributed computing environments where certain tasks are performed by remote processing devices that are linked through a communications network. In a distributed computing environment, program modules can be located in both local and remote memory storage devices.

Referring to FIG. 11, there is illustrated a schematic block diagram of a computing environment 1100 in accordance with this disclosure in which the subject systems (e.g., system 110 and the like), methods and computer readable media can be deployed. The computing environment 1100 includes one or more client(s) 1102 (e.g., laptops, smart phones, PDAs, media players, computers, portable electronic devices, tablets, and the like). The client(s) 1102 can be hardware and/or software (e.g., threads, processes, computing devices). The computing environment 1100 also includes one or more server(s) 1104. The server(s) 1104 can also be hardware or hardware in combination with software (e.g., threads, processes, computing devices). The servers 1104 can house threads to perform transformations by employing aspects of this disclosure, for example. In various embodiments, one or more components, devices, systems, or subsystems of system 110 can be deployed as hardware and/or software at a client 1102 and/or as hardware and/or software deployed at a server 1104. One possible communication between a client 1102 and a server 1104 can be in the form of a data packet transmitted between two or more computer processes wherein the data packet may include healthcare related data, training data, AI models, input data for the AI models, encrypted output data generated by the AI models, and the like. The data packet can include a metadata, e.g., associated contextual information, for example. The computing environment 1100 includes a communication framework 1106 (e.g., a global communication network such as the Internet, or mobile network(s)) that can be employed to facilitate communications between the client(s) 1102 and the server(s) 1104.

Communications can be facilitated via a wired (including optical fiber) and/or wireless technology. The client(s) 1102 include or are operatively connected to one or more client data store(s) 1108 that can be employed to store information local to the client(s) 1102 (e.g., associated contextual information). Similarly, the server(s) 1104 are operatively include or are operatively connected to one or more server data store(s) 1110 that can be employed to store information local to the servers 1104.

In one embodiment, a client 1102 can transfer an encoded file, in accordance with the disclosed subject matter, to server 1104. Server 1104 can store the file, decode the file, or transmit the file to another client 1102. It is to be appreciated, that a client 1102 can also transfer uncompressed file to a server 1104 can compress the file in accordance with the disclosed subject matter. Likewise, server 1104 can encode video information and transmit the information via communication framework 1106 to one or more clients 1102.

While the subject matter has been described above in the general context of computer-executable instructions of a computer program product that runs on a computer and/or computers, those skilled in the art will recognize that this disclosure also can or can be implemented in combination with other program modules. Generally, program modules include routines, programs, components, data structures, etc. that perform particular tasks and/or implement particular abstract data types. Moreover, those skilled in the art will appreciate that the inventive computer-implemented methods can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments where tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “subsystem” “platform,” “layer,” “gateway,” “interface,” “service,” “application,” “device,” and the like, can refer to and/or can include one or more computer-related entities or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration and are intended to be non-limiting. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of entity equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim. The descriptions of the various embodiments have been presented for purposes of illustration, but are not intended to be exhaustive or limited to the embodiments disclosed. Many modifications and variations can be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system, comprising:
a memory that stores computer executable components; and
a processor that executes the computer executable components stored in the memory, wherein the computer executable components comprise:
a machine learning component that receives historical clinical data messages converted from one or more first native formats to a target format via a mapping function that maps different sets of historical data elements included in the historical clinical data messages into defined data description paths, and wherein the machine learning component trains anomaly detection models for each of the defined data description paths using machine learning to characterize normal characteristics of the different sets of historical data elements for each of the defined data description paths; and
an anomaly detection component that receives new clinical data messages converted from the one or more first native formats or one or more second native formats to the target format via the mapping function and detects the abnormal characteristics of different sets of new data elements mapped from the new clinical data messages for corresponding data description paths of the defined data description paths using the anomaly detection models.

2. The system of claim 1, wherein the historical clinical data messages and the new clinical data messages were generated by one or more clinical information resources associated with a same hospital system.

3. The system of claim 1, wherein the historical clinical data messages were generated by one or more first clinical information resources associated with a first same hospital system and wherein the new clinical data messages were generated by one or more second clinical information resources associated with a second same hospital system.

4. The system of claim 1, wherein the anomaly detection component applies respective anomaly detection models of the anomaly detection models for the corresponding data description paths to the different sets of new data elements respectively mapped to the corresponding data description paths and generates anomaly scores for each of the corresponding data description paths that represent an amount or severity of the abnormal characteristics associated with each of the corresponding data description paths, and wherein the computer executable components further comprise:
an alert component that generates an integration error alert for any of the corresponding data description paths whose anomaly score exceeds a threshold anomaly score;
a reporting component that generates integration report data identifying the anomaly scores for the corresponding data description paths and identifying any of the corresponding data description paths associated with an integration error alert; and
a rendering component that presents the integration report data via a graphical user interface.

5. The system of claim 4, wherein the reporting component further identifies one or more data samples for the corresponding data description paths and provides links to the one or more data samples within the integration report data.

6. The system of claim 1, wherein the anomaly detection component applies respective anomaly detection models of the anomaly detection models for the corresponding data description paths to the different sets of new data elements respectively mapped to the corresponding data description paths and generates anomaly scores for each of the corresponding data description paths that represent an amount or severity of the abnormal characteristics associated with each of the corresponding data description paths, and wherein the computer executable components further comprise:
an alert component that generates an integration error alert for any of the corresponding data description paths whose anomaly score exceeds a threshold anomaly score; and
a reporting component that reports that the integration error alert in real-time in response to generation thereof.

7. The system of claim 4, wherein the computer executable components further comprise:
a feedback component that facilitates receiving user feedback regarding accuracy of the anomaly scores, and wherein the machine learning component further retrains one or more of the anomaly detection models based on the user feedback.

8. The system of claim 1, wherein the abnormal characteristics comprise abnormal values for the different sets of new data elements and wherein the machine learning comprises learning normal values for the different sets of historical data elements based on the historical clinical data messages.

9. The system of claim 8, wherein the each of the anomaly detection models comprises variational autoencoders and wherein the machine learning comprises training the variational autoencoders to learn the normal values for the different sets of historical data elements based on the historical clinical data messages.

10. The system of claim 9, wherein the anomaly detection models comprise variational autoencoders and wherein the machine learning further comprises training one or more of the variational autoencoders to learn a conditional relationship between the normal values for one or more pairs of the defined data description paths.

11. The system of claim 1, wherein the target format comprises the Fast Healthcare Interoperability Resources (FHIR) format and wherein each of the defined data description paths correspond to a different FHIR key.

12. The method, comprising:
receiving, by a system comprising a processor, historical clinical data messages converted from one or more first native formats to a target format via a mapping function that maps different sets of historical data elements included in the historical clinical data messages into defined data description paths;
training, by the system, anomaly detection models for each of the defined data description paths using machine learning to characterize normal characteristics of the different sets of historical data elements for each of the defined data description paths;
receiving, by the system, new clinical data messages converted from the one or more first native formats or one or more second native formats to the target format via the mapping function; and
detecting, by the system, abnormal characteristics of different sets of new data elements mapped from the new clinical data messages for corresponding data description paths of the defined data description paths using the anomaly detection models.

13. The method of claim 12, wherein the historical clinical data messages and the new clinical data messages were generated by one or more clinical information resources associated with a same hospital system.

14. The method of claim 12, wherein the historical clinical data messages were generated by one or more first clinical information resources associated with a first same hospital system and wherein the new clinical data messages were generated by one or more second clinical information resources associated with a second same hospital system.

15. The method of claim 12, wherein the detecting comprises:
applying, by the system, respective anomaly detection models of the anomaly detection models for the corresponding data description paths to the different sets of new data elements respectively mapped to corresponding data description paths for the new clinical data messages;
generating, by the system, anomaly scores for each of the corresponding data description paths that represent an amount or severity of the abnormal characteristics associated with each of the corresponding data description paths; and
generating, by the system, an integration error alert for any of the corresponding data description paths whose anomaly score exceeds a threshold anomaly score.

16. The method of claim 15, further comprising:
generating, by the system, integration report data identifying the anomaly scores for each of the corresponding data description paths and identifying any of the corresponding data description paths associated with an integration error alert; and
presenting, by the system, the integration report data via a graphical user interface.

17. The method of claim 16, further comprising:
receiving, by the system, user feedback regarding accuracy of the anomaly scores; and
retraining, by the system, one or more of the anomaly detection models based on the user feedback.

18. The method of claim 12, wherein the anomaly detection models comprise variational autoencoders and wherein the machine learning comprises at least one of:
training, by the system, one or more of the variational autoencoders to learn the normal values for the different sets of historical data elements based on the historical clinical data messages; or
training, by the system, one or more of the variational autoencoders to learn a conditional relationship between the normal values for one or more pairs of the defined data description paths.

19. A non-transitory machine-readable storage medium, comprising executable instructions that, when executed by a processor, facilitate performance of operations, comprising:
receiving historical clinical data messages converted from one or more first native formats to a target format via a mapping function that maps different sets of historical data elements included in the historical clinical data messages into defined data description paths;
training anomaly detection models for each of the defined data description paths using machine learning to characterize normal characteristics of the different sets of data elements for each of the defined data description paths;
receiving new clinical data messages converted from the one or more first native formats or one or more second native formats to the target format via the mapping function; and
detecting abnormal characteristics of different sets of new data elements mapped from the new clinical data messages for corresponding data description paths of the defined data description paths using the anomaly detection models.

20. The non-transitory machine-readable storage medium of claim 19, wherein the detecting comprises:
applying respective anomaly detection models of the anomaly detection models for the corresponding data description paths to the different sets of new data elements respectively mapped to corresponding data description paths for the new clinical data messages;
generating anomaly scores for each of the corresponding data description paths that represent an amount or severity of the abnormal characteristics associated with each of the corresponding data description paths; and
generating an integration error alert for any of the corresponding data description paths whose anomaly score exceeds a threshold anomaly score.
